(19) United States
(12) Patent Application Publication
(10) Pub. No.: US 2026/0263080 A1
(43) Pub. Date: Sep. 10, 2026

(54) DEVICES, SYSTEM AND METHODS FOR APPLYING MULTIPLE CLIPS TO TISSUE

(71) Applicant: INTUITIVE SURGICAL OPERATIONS, INC., Sunnyvale, CA (US)

(72) Inventor: Ronald G. LITKE, Sandy Hook, CT (US)

(73) Assignee: INTUITIVE SURGICAL OPERATIONS, INC., Sunnyvale, CA (US)

(21) Appl. No.: 19/489,259

(22) PCT Filed: Jun. 3, 2024
(86) PCT No.: PCT/US2024/032233

§ 371 Date: Dec. 1, 2025

Related U.S. Application Data
(60) Provisional application No. 63/505,735, filed on Jun. 2, 2023.
(60) Provisional application No. 63/505,738, filed on Jun. 2, 2023.
(60) Provisional application No. 63/505,740, filed on Jun. 2, 2023.
(60) Provisional application No. 63/505,742, filed on Jun. 2, 2023.
(60) Provisional application No. 63/505,870, filed on Jun. 2, 2023.
(60) Provisional application No. 63/505,875, filed on Jun. 2, 2023.

(51) Int. Cl. A61B 17/122; A61B 17/00; A61B 17/128; A61B 34/30

(52) U.S. Cl. CPC A61B 17/122; A61B 17/1285; A61B 34/30; A61B 2017/00477

(57) ABSTRACT

A surgical instrument for applying surgical clips to tissue comprises a shaft comprising a proximal end and a distal end, a wrist mechanism coupled to the distal end of the shaft and configured to articulate relative to the shaft in one or more degrees of freedom and an end effector supported by the wrist mechanism. The end effector comprises a pair of opposing jaw members configured to move relative to each other between open and closed positions. A path configured to receive and permit advancement of a medical clip into engagement with the jaw members is defined through the wrist mechanism and end effector. The instrument allows a surgeon, for example, to deliver one or more surgical clips onto tissue or vessels without having to exchange the instrument or to change the orientation of the jaws during clip advancement, which reduces disruption to the surgeon's workflow.

### CROSS-REFERENCE TO RELATED APPLICATIONS

This application claims the benefit of the following U.S. Provisional Applications: (1) Ser. No. 63/505,738, filed Jun. 2, 2023; (2) Ser. No. 63/505,740, filed Jun. 2, 2023, (3) Ser. No. 63/505,742, filed Jun. 2, 2023; (4) Ser. No. 63/505,870, filed Jun. 2, 2023; (5) Ser. No. 63/505,875, filed Jun. 2, 2023; and (6) Ser. No. 63/505,735, filed Jun. 2, 2023, the complete disclosures of which are incorporated herein by reference for all purposes.

### BACKGROUND

This description generally relates to endoscopic surgical instruments for dissecting, occluding and/or sealing tissue, and more particularly to endoscopic surgical instruments capable of applying multiple clips to vessels and/or tissue.

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, is reduced post-operative hospital recovery times. The average hospital stay for a standard open surgery is typically significantly longer than the average stay for an analogous minimally invasive surgery (MIS). Thus, increased use of MIS could save millions of dollars in hospital costs each year. While many of the surgeries performed each year in the United States could potentially be performed in a minimally invasive manner, only a portion of the current surgeries uses these advantageous techniques due to limitations in minimally invasive surgical instruments and the additional surgical training involved in mastering them.

Improved surgical instruments such as tissue access, navigation, dissection and sealing instruments have enabled MIS to redefine the field of surgery. These instruments allow surgeries and diagnostic procedures to be performed with reduced trauma to the patient. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for laparoscopic instruments.

Laparoscopic surgical instruments generally include an endoscope (e.g., laparoscope) for viewing the surgical field and tools for working at the surgical site. The working tools are typically similar to those used in conventional (open) surgery, except that the working end or end effector of each tool is separated from its handle by an extension tube (also known as, e.g., an instrument shaft or a main shaft). The end effector can include, for example, a clamp, grasper, scissor, stapler, cautery tool, linear cutter, or needle holder.

To perform surgical procedures, the surgeon passes working tools through cannula sleeves to an internal surgical site and manipulates them from outside the abdomen. The surgeon views the procedure from a monitor that displays an image of the surgical site taken from the endoscope. Similar endoscopic techniques are employed in, for example, arthroscopy, retroperitoneoscopy, pelviscopy, nephroscopy, cystoscopy, cisternoscopy, sinoscopy, hysteroscopy, urethroscopy, and the like.

Minimally invasive telesurgical robotic systems are being developed to increase a surgeon's dexterity when working on an internal surgical site, as well as to allow a surgeon to operate on a patient from a remote location (outside the sterile field). In a telesurgery system, the surgeon is often provided with an image of the surgical site at a control console. While viewing a three dimensional image of the surgical site on a suitable viewer or display, the surgeon performs the surgical procedures on the patient by manipulating master input or control devices of the control console, which in turn control motion of the servo-mechanically operated slave instruments.

The servomechanism used for telesurgery will often accept input from two master controllers (one for each of the surgeon's hands) and may include two or more robotic arms on each of which a surgical instrument is mounted. Operative communication between master controllers and associated robotic arm and instrument assemblies is typically achieved through a control system. The control system typically includes at least one processor that relays input commands from the master controllers to the associated robotic arm and instrument assemblies and back from the instrument and arm assemblies to the associated master controllers in the case of, for example, force feedback or the like. One example of a robotic surgical system is the DA VINCI™ system commercialized by Intuitive Surgical, Inc. of Sunnyvale, California.

A variety of structural arrangements have been used to support the surgical instrument at the surgical site during robotic surgery. The driven linkage or “slave” is often called a robotic surgical manipulator, and exemplary linkage arrangements for use as a robotic surgical manipulator during minimally invasive robotic surgery are described in U.S. Pat. No. 7,594,912 (filed Sep. 30, 2004), U.S. Pat. No. 6,758,843 (filed Apr. 26, 2002), U.S. Pat. No. 6,246,200 (filed Aug. 3, 1999), and U.S. Pat. No. 5,800,423 (filed Jul. 20, 1995), the full disclosures of which are incorporated herein by reference in their entirety for all purposes. These linkages often manipulate an instrument holder to which an instrument having a shaft is mounted. Such a manipulator structure can include a parallelogram linkage portion that generates motion of the instrument holder that is limited to rotation about a pitch axis that intersects a remote center of manipulation located along the length of the instrument shaft. Such a manipulator structure can also include a yaw joint that generates motion of the instrument holder that is limited to rotation about a yaw axis that is perpendicular to the pitch axis and that also intersects the remote center of manipulation. By aligning the remote center of manipulation with the incision point to the internal surgical site (for example, with a trocar or cannula at an abdominal wall during laparoscopic surgery), an end effector of the surgical instrument can be positioned safely by moving the proximal end of the shaft using the manipulator linkage without imposing potentially hazardous forces against the abdominal wall. Alternative manipulator structures are described, for example, in U.S. Pat. No. 6,702,805 (filed Nov. 9, 2000), U.S. Pat. No. 6,676,669 (filed Jan. 16, 2002), U.S. Pat. No. 5,855,583 (filed Nov. 22, 1996), U.S. Pat. No. 5,808,665 (filed Sep. 9, 1996), U.S. Pat. No. 5,445,166 (filed Apr. 6, 1994), and U.S. Pat. No. 5,184,601 (filed Aug. 5, 1991), the full disclosures of which are incorporated herein by reference in their entirety for all purposes.

During the surgical procedure, the telesurgical system can provide mechanical actuation and control of a variety of surgical instruments or tools having end effectors that perform various functions for the surgeon, for example, holding or driving a needle, grasping a blood vessel, dissecting tissue, or the like, in response to manipulation of the master input devices. Manipulation and control of these end effectors is a particularly beneficial aspect of robotic surgical systems. Such mechanisms should be appropriately sized for use in a minimally invasive procedure and relatively simple in design to reduce possible points of failure. In addition, such mechanisms should provide an adequate range of motion to allow the end effector to be manipulated in a wide variety of positions.

Endoscopic surgical clip appliers are used for a number of minimally invasive or endoscopic surgical procedures to occlude, ligate and/or seal vessels and tissue. Applying surgical clips usually involves compressing the clip over the surgical site, such as a blood vessel, duct, or similar tissue structure. Once applied to the tissue structure, the compressed surgical clip terminates the flow of fluid therethrough.

Conventional surgical clips are designed to be compressed into a latched or locked position around a grasped vessel or other grasped tissue. Typically, the surgical instrument includes jaws that can be closed to engage bosses formed on the clips. These bosses are forced inwardly about a hinge section causing the first and second legs of the clip being applied to close around the grasped vessel. The tip section of the second leg then begins to contact a hook section. Upon further closing of the jaws, the tip section snaps into and is conformably seated in the latching recess, at which point the clip is secured into a latched condition.

Certain endoscopic surgical clip appliers include a surgical instrument having an end effector with movable jaws and a single clip that is installed within the end effector. These instruments are limited to a single discharge per instrument. In other words, once a clip has been discharged and applied to tissue, the surgeon must remove the surgical instrument from the cannula and manually reload a new clip into the instrument, or use a completely different surgical clip applier (i.e., a new instrument).

Other laparoscopic clip appliers have been developed with a cartridge that may be preloaded with about 2-10 clips. These clip appliers, however, are typically disposable and designed to be discarded after a procedure. Some existing endoscopic surgical clip appliers are “straight” or “non-wristed” instruments that do not allow the user to change the orientation of the jaws relative to the shaft of the instrument during, or after, clip advancement.

Multi-fire instruments are designed to hold a plurality of medical clips that are sequentially supplied to the jaw members of the instrument intraoperatively (i.e., without having to withdraw the instrument from within a body cavity). Challenges associated with such instruments include: (1) accurately and securely advancing clips to jaw members and holding clips within the jaw members for subsequent application; (2) generating sufficient force at the jaw members to apply and close the clip; and (3) holding and advancing multiple clips while permitting articulation of the jaw members relative to each other to open and close.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

In one aspect, a surgical instrument for applying surgical clips to tissue comprises an elongate shaft having a longitudinal axis, an end effector on a distal end of the shaft including first and second jaws, an articulation assembly coupled to the first and second jaws and configured to move the first and second jaws between an open position and a closed position and a drive member configured to translate longitudinal through the articulation assembly to deliver one or more surgical clips to the first and second jaws.

In various embodiments, the articulation assembly comprises a pulley rotatable about an axis substantially perpendicular to the longitudinal axis, and an elongate drive member, such as a cable or band, around the pulley and coupled to the first and second jaws. Rotation of the pulley in a first direction causes the first and second jaws to move away from each other. Rotation of the pulley in a second direction causes the first and second jaws to move towards each other. In an exemplary embodiment, the first and second jaws each comprise a curved drive surface and the band extends along the curved drive surfaces.

The articulation assembly allows a surgeon, for example, to deliver one or more surgical clips onto tissue or vessels without having to change the orientation of the jaws during clip advancement, which reduces disruption to the surgeon's workflow. Providing at least one degree of rotational movement relative to the shaft enables the end effector to correspond with at least a portion of the natural action of a surgeon's wrist, thereby facilitating placement of the jaws in the optimal location for performing the sealing/occluding function, particularly in a laparoscopic procedure wherein the instrument has been inserted through a small entry point into the abdominal cavity.

In embodiments, the articulation assembly comprises first and second extension members each having an opening. Each of the first and second jaws comprises an axle disposed within one of the openings. Each of the jaws may further comprise a lateral surface with an opening. The axle of the first jaw extends through the opening in the second jaw and the axle of the second jaw extends through the opening in the first jaw.

In various embodiments, the drive member comprises a distal component for removably coupling to a surgical clip. The first and second jaws may include first and second guide tracks, respectively, for receiving protrusions on the surgical clip to guide the surgical clip to the jaws. The surgical clip may include first and second arms, and at least one of the first and second jaws comprises an engagement element for securing at least one of the first and second arms of the clip to said at least one of the first and second jaws.

The drive member retains and controls the distal ends of the clip arms, which allows the drive member to position the clip arms within the jaws of the end effector and to retain the clip while the end effector is opened and closed and/or articulated relative to the shaft of the instrument. This allows the surgeon to fully open the clips after they have been advanced into the jaws so that they can be effectively positioned around a target vessel or tissue. In addition, this allows the surgeon to reposition the jaws relative to the shaft after the clip has been advanced into the jaws.

In embodiments, the first and second jaws each comprise a guide track extending from a wrist member to a distal end of the jaws. The guide tracks facilitate the advancement of the clip and the engagement elements of the drive member through the jaws and into position such that the jaws can open and/or close the clip. The end effector may further comprise first and second flexible bands, cables or ribbons extending from the wrist member to a distal end of each of the first and second jaws. The first and second ribbons comprise at least a semi-flexible material configured to bend when the first and second jaws articulate about the wrist member relative to the shaft. The ribbons constrain the drive member and the clip as they are advanced into the jaws and ensure that they stay within the guide tracks in the event that the jaws are articulated during clip advancement.

In various embodiments, the instrument further comprises a wrist mechanism or assembly comprising an internal channel for advancing the drive member therethrough. This allows a user to deliver surgical clips onto tissue or vessels through the wrist member so that the end effector can be rotated in at least two axes relative to the shaft, further increasing the ability of the surgeon to reposition the jaws relative to the target vessel or tissue.

The articulation assembly may also be disposed laterally outward from the first and second jaws relative to the longitudinal axis of the shaft. The instrument allows a surgeon, for example, to deliver one or more surgical clips onto tissue or vessels without having to exchange the instrument or to change the orientation of the jaws during clip advancement, which reduces disruption to the surgeon's workflow.

In various embodiments, the articulation assembly comprises first and second pulleys disposed laterally outward from the internal channel and rotatable about an axis substantially perpendicular to the longitudinal axis. A first band extends around at least a portion of the first pulley and a second band extends around at least a portion of the second pulley. The bands are operable to rotate the pulleys upon longitudinal translation of a proximal end portion of the bands.

In various embodiments, rotation of the first pulley in a first direction causes the first jaw to move away from the second jaw and rotation of the second pully in a second direction opposite to the first direction causes the second jaw to move away from the first jaw. Rotation of the first pulley in the second direction causes the first jaw to move towards from the second jaw and rotation of the second pully in the first direction causes the second jaw to move towards the first jaw. Thus, rotation of the first and second pulleys in opposite directions causes the first and second jaws to move between the open and closed positions.

In various embodiments, rotation of the first and second pulleys in the same direction causes the first and second jaws to articulate relative to the shaft about an axis substantially perpendicular to the longitudinal axis. In an exemplary embodiment, this axis is a pitch axis relative to the shaft.

In various embodiments, the articulation assembly further comprises a first link having a proximal end coupled to the first pulley and a distal end coupled to the first jaw, and a second link having a proximal end coupled to the second pulley and a distal end coupled to the first jaw. The first and second links are disposed laterally outward from the first and second jaws.

In various embodiments, the second link has a longer length than the first link. The first link may be substantially linear and the second link may comprise a distal linear portion and a proximal curved portion. The assembly may further comprise a pivot pin extending through the first jaw and coupled to the first and second links.

In various embodiments, the assembly further comprises a third link having a proximal end coupled to the second pulley and a distal end coupled to the second jaw, and a fourth link having a proximal end coupled to the first pulley and a distal end coupled to the second jaw. The third and fourth links are disposed laterally outward from the first and second jaws. The fourth link may have a longer length than the third link. The third link may be substantially linear and the fourth link may comprise a distal linear portion and a proximal curved portion.

In various embodiments, the instrument further comprises an actuator extending through the articulation assembly lateral of the drive member and being configured to move the jaws between the open and closed positions.

In another aspect, a surgical instrument for applying surgical clips to tissue comprises an elongate shaft having a longitudinal axis, an end effector on a distal end of the shaft including first and second jaws, an articulation assembly coupled to the first and second jaws and configured to move the first and second jaws between an open position and a closed position, the articulation assembly comprising an internal longitudinal channel and a drive member configured to translate longitudinal through the internal channel of the articulation assembly to deliver one or more surgical clips to the first and second jaws.

In various embodiments, the articulation assembly comprises first and second cam pins coupled to the first and second jaws, respectively. The articulation assembly may further comprise first and second connection portions coupled to either side of the first jaw. The first and second connection portions each comprising a cam slot for receiving the first cam pin. In an exemplary embodiment, the cam slots are non-linear.

In various embodiments, the articulation assembly further comprises third and fourth connection portions coupled to either side of the second jaw. The third and fourth connection portions each comprising a cam slot for receiving the second cam pin. The first, second, third and fourth connection portions are disposed laterally outward from the first and second jaws relative to the longitudinal axis.

In various embodiments, the instrument further comprises first and second plates coupled to the shaft and disposed laterally outward from the first and second jaws. The first and third connection portions are pivotally coupled to the first plate and the second and fourth connection portions are pivotally coupled to the second plate.

In various embodiments, the instrument further a wrist assembly coupling the end effector to the shaft. The wrist assembly comprising an internal channel for receiving the drive member and first and second links coupled to each other by a joint. In one such embodiment, the wrist assembly comprises first and second links pivotally coupled to each other about a joint for rotating the end effector about a first axis perpendicular to the longitudinal axis (e.g., a pitch or yaw axis). The first and second links and the joint are disposed laterally outward of the internal channel of the wrist assembly.

In various embodiments, the first link is coupled to the end effector and the second link is disposed proximal of the first link. The wrist assembly further comprising a third link pivotally coupled to the second link about a second joint for rotating the end effector about a second axis perpendicular to the longitudinal axis (e.g., a pitch or yaw axis). The third link and the second joint are disposed laterally outward of the internal channel of the wrist assembly.

In various embodiments, the drive member comprises a distal component for removably coupling to a surgical clip. The first and second jaws may include first and second guide tracks, respectively, for receiving protrusions on the surgical clip to guide the surgical clip to the jaws. The surgical clip may include first and second arms, and at least one of the first and second jaws comprises an engagement element for securing at least one of the first and second arms of the clip to said at least one of the first and second jaws.

In another aspect, a surgical instrument for applying surgical clips to tissue comprises an elongate shaft having a longitudinal axis, an end effector on a distal end of the shaft including first and second jaws, a wrist assembly coupling the end effector to the shaft for rotating the end effector about an axis substantially perpendicular to the longitudinal axis and a drive member configured to translate longitudinal through the wrist assembly to deliver one or more surgical clips to the first and second jaws. The instrument allows a surgeon, for example, to deliver one or more surgical clips onto tissue or vessels while changing the orientation of the jaws during clip advancement, which reduces disruption to the surgeon's workflow.

In various embodiments, the wrist assembly comprises an internal channel and a first link extending circumferentially around the internal channel from a first end to a second end. The first end is proximal to the second end. The wrist assembly further comprises a second link extending circumferentially around the internal channel from a first end to a second end. The first end is proximal to the second end.

In various embodiments, the first end of the first link is substantially circumferentially aligned with the second end of the second link and the second end of the first link is substantially circumferentially aligned with the first end of the second link. The first and second links are movable to allow rotation of the end effector relative to the shaft.

In various embodiments, the wrist assembly comprises an internal channel first and second pulleys disposed laterally outward from the internal channel and rotatable about an axis substantially perpendicular to the longitudinal axis. The wrist may further comprise a first band around at least a portion of the first pulley and a second band around at least a portion of the second pulley, the bands operable to rotate the pulleys upon longitudinal translation of a proximal end portion of the bands.

In various embodiments, the instrument further comprises an articulation assembly coupled to the first and second jaws and configured to move the first and second jaws between an open position and a closed position. The articulation assembly comprises an internal channel and the drive member is configured to translate longitudinally through the internal channel.

In various embodiments, the instrument comprises an actuator extending through the wrist assembly lateral of the drive member and being configured to move the jaws between the open and closed positions.

In various embodiments, the drive member comprises a distal component for removably coupling to a surgical clip. The first and second jaws may include first and second guide tracks, respectively, for receiving protrusions on the surgical clip to guide the surgical clip to the jaws. The surgical clip may include first and second arms, and at least one of the first and second jaws comprises an engagement element for securing at least one of the first and second arms of the clip to said at least one of the first and second jaws.

It is to be understood that both the foregoing general description and the following detailed description are exemplary and explanatory only and are not restrictive of the description. Additional features will be set forth in part in the description which follows or may be learned by practice of the description.

### BRIEF DESCRIPTION OF THE DRAWINGS

The above and other aspects, features, and advantages of the present surgical instruments will become more apparent in light of the following detailed description when taken in conjunction with the accompanying drawings in which:

FIG. 1 is a schematic, perspective view of an embodiment of a medical instrument for applying medical clips;

FIG. 2 is a diagrammatic view of an embodiment of teleoperated, computer-assisted medical system;

FIG. 3A is a partial, perspective view of an embodiment of a distal end portion of an instrument for applying medical clips;

FIG. 3B is a partial, cross-sectional view of the distal end portion of the instrument of FIG. 3A taken through section 3B-3B;

FIG. 3C is a detailed view of portion 3C of FIG. 3A showing the jaw members of the instrument in partially closed position and holding a medical clip.

FIGS. 4A and 4B are longitudinal cross-sectional views of a distal portion of an instrument for applying medical clips comprising the end effector of FIGS. 3A-3C coupled to the instrument shaft via a wrist mechanism comprising a series of links coupled at joints.

FIGS. 5A and 5B are schematic views of a crossed four-bar linkage, in differing states of articulation, that can be used as a wrist mechanism in accordance with various embodiments;

FIGS. 6A and 6B show an embodiment of the distal portion of an instrument for applying medical clips comprising the end effector of FIGS. 3A-3C coupled to a shaft by crossed four-bar linkage wrist mechanism, with the wrist being in a neutral state in FIG. 6A and in an articulated state in FIG. 6B;

FIG. 7 is a perspective view of an embodiment of a distal end portion of an instrument for applying medical clips using an eight-bar linkage as a wrist mechanism;

FIG. 8A is a perspective view of another embodiment of a distal end portion of an instrument for applying medical clips comprising a pulley and linkage system wrist mechanism shown in a neutral state of articulation;

FIG. 8B is a perspective view of the distal end portion of the instrument of FIG. 8A depicting the wrist mechanism articulated in pitch or yaw depending on the orientation of the device relative to the user;

FIG. 9 is an isolated perspective view of a jaw member and portions of the pulley and linkage system wrist mechanism of FIGS. 8A and 8B;

FIGS. 10A and 10B are perspective, partially transparent views of a distal end portion of an instrument for applying medical clips comprising a pulley and linkage system wrist mechanism according to another embodiment;

FIGS. 10C and 10D are views similar to FIGS. 10A and 10B except showing articulation of the end effector in pitch or yaw depending on the orientation of the device relative to the user;

FIG. 11 is a side perspective view of another embodiment of a distal end portion of an instrument for applying medical clips comprising a pulley and linkage system wrist mechanism;

FIGS. 12A-12C are partial, sectional views of another embodiment of a distal end portion of an instrument for applying medical clips, showing various states of articulation in pitch or yaw depending on the orientation of the device relative to the user;

FIG. 13 is a partial, sectional view of yet another embodiment of a distal end portion of an instrument for applying medical clips;

FIG. 14 is a partial, sectional view of an embodiment of a shaft, wrist mechanism, and clevis of an instrument for applying medical clips;

FIG. 15A is perspective view of another embodiment of a distal end portion of an instrument for applying medical clips comprising a wrist mechanism configured to articulate in pitch and yaw, with the wrist mechanism shown in a neutral state of articulation;

FIG. 15B is a perspective view of the distal end portion of the instrument of FIG. 13A depicting the wrist articulated in pitch and yaw;

FIG. 16 is the view of FIG. 15A, but with the pitch wrist mechanism shown partially articulated and exploded;

FIG. 17 view of FIG. 15A, but with the pitch wrist mechanism shown partially articulated and partially transparent to better show actuation members; and

FIG. 18 is a partial, longitudinal sectional view of FIG. 15A;

FIG. 19 is a perspective view of another embodiment of a distal end portion of an instrument for applying medical clips;

FIG. 20 is an exploded view of the distal end portion of FIG. 19; and

FIG. 21 is a cross-sectional view of the distal end portion of FIG. 19.

### DETAILED DESCRIPTION

Particular embodiments of the present surgical instruments are described hereinbelow with reference to the accompanying drawings; however, it is to be understood that the disclosed embodiments are merely exemplary and may be embodied in various forms. Therefore, specific structural and functional details disclosed herein are not to be interpreted as limiting, but merely as a basis for the claims and as a representative basis for teaching one skilled in the art to variously employ the devices herein in virtually any appropriately detailed structure. Well-known functions or constructions are not described in detail to avoid obscuring the present description in any unnecessary detail. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

Various embodiments of medical instruments are provided that configured to apply medical clips that provide for a relatively small overall size profile conducive for minimally invasive medical procedures (e.g., endoscopic, laparoscopic, etc.), while addressing various challenges facing conventional medical clip applier instruments. For example, various embodiments in accordance with the present disclosure provide medical instruments configured to apply medical clips that provide for a relatively small overall size profile conducive for minimally invasive medical procedures (e.g., endoscopic, laparoscopic, etc.) while also having sufficient space to hold and apply multiple clips intraoperatively (i.e., without having to remove and reload a clip into the instrument). The present disclosure further contemplates various embodiments of such medical instruments that enable firing and application of multiple clips intraoperatively while providing the ability to securely load and apply the clips, for example, advancing the clip from proximal to the jaw members and then to a state held by the jaw members for subsequent application. Embodiments of the present disclosure further contemplate the ability to advance clips from proximal to a wrist mechanism that couples the jaw members to the instrument shaft.

Various aspects further provide for medical instruments that can apply multiple clips intraoperatively while allowing articulation of the jaw members via a wrist mechanism relative to the shaft in one or more degrees of freedom (e.g., in pitch and/or yaw) and with enough space between the jaw members to receive a clip and position the clip around the objected (e.g., tissue, body vessel, etc.) to be clipped, and providing sufficient clamping force to close and latch the clip. Embodiments described herein permit the jaw members to open through a range of motion from a closed state to an angle between the jaw members ranging from 20-45 degrees, for example, from 30-35 degrees, and the jaws may be configured to articulate in pitch and/or yaw relative to the instrument shaft through a range of at least +/−45 degrees, such as for example, through a range of +/−60 degrees, or for example through a range of +/−65 degrees.

While the following is presented with respect to surgical instruments that are compatible with surgical clip cartridges, it should be understood that certain features of the presently described surgical instruments may be readily adapted for use in any type of surgical clamping, cutting, ligating, dissecting, clipping, cauterizing, suturing and/or sealing instrument, whether or not the surgical instrument applies a clip or other type of fastener. Additionally, the features of the presently described surgical ligating instruments may be readily adapted for use in surgical instruments that are actuated using any technique within the purview of those skilled in the art, such as, for example, manually activated surgical instruments, powered surgical instruments (e.g., electro-mechanically powered instruments), robotic surgical instruments, and the like.

The devices described herein, or certain components of the devices, may also be incorporated into a variety of different surgical instruments, such as those described in commonly assigned, co-pending U.S. patent application Ser. Nos. 16/205,128, 16/427,427, 16/678,405, 16/904,482, 17/081,088 and 17/084,981 and International Patent Nos. PCT/US2019/107646, PCT/US2019/019501, PCT/US2019/062344, PCT/US2020/54568, PCT/US2019/064861, PCT/US2019/062768, PCT/2020/025655, PCT/US2020/056979, PCT/2019/066513, PCT/US2020/020672, PCT/US2019/066530 and PCT/US2020/033481, the complete disclosures of which are incorporated by reference herein in their entirety for all purposes as if copied and pasted herein.

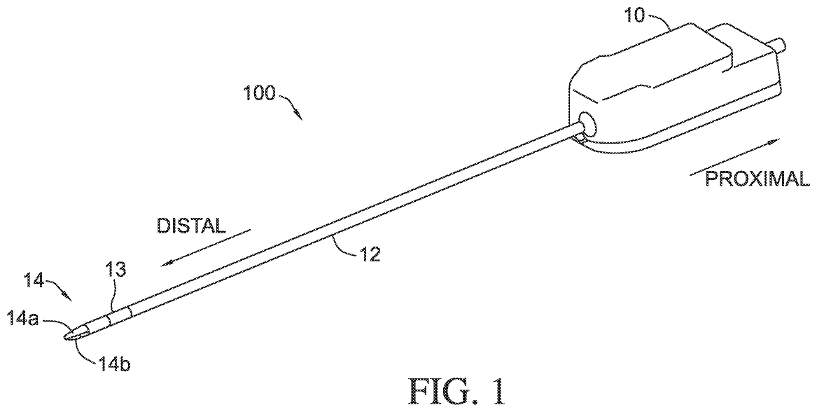

FIG. 1 is a schematic perspective view of an embodiment of a medical instrument configured to apply multiple clips without the need to withdraw the instrument from a remote worksite to reload the instrument. The directions “proximal” and “distal” are used herein to define the directions as shown in FIG. 1, with distal generally being in a direction further along a kinematic arm or closest to the worksite in the intended operational use of the instrument 100, for example, in use for applying medical clips during a medical procedure. As shown in FIG. 1, the instrument 100 generally includes an instrument shaft 12 extending between and coupling a force transmission mechanism 10 at a proximal end portion of the instrument 100 and an end effector 14 disposed at a distal end portion of the instrument 100. The force transmission mechanism 10 can have various inputs configured to be manually actuated or actuated through engagement of a motorized mechanism, which inputs in turn drive various drive members (e.g., pulleys, capstans, gears etc.) of the force transmission mechanism, with the drive members being coupled with and transmitting force via actuation members, such as cables, rods, etc., to actuate distal components (e.g., joints, end effector, etc.) to which the actuation members are also coupled. Those of ordinary skill in the art are generally familiar with such components and they are thus not described in detail here. In the exemplary embodiment shown in FIG. 1, the medical instrument 100 also includes an optional articulatable wrist mechanism 13 mounted at the distal end portion of the shaft 12 to support the end effector 14 and change its orientation via articulation of the wrist mechanism 13 with reference to the shaft's 12 longitudinal axis. In various embodiments, as will be explained further below, the wrist mechanism 13 may be configured to articulate the end effector in pitch and/or yaw relative to the shaft 12, with pitch and yaw being articulation directions about orthogonal axes, such as an axis parallel to a longitudinal axis of the shaft and an axis perpendicular to the longitudinal axis of the shaft. The yaw axis will be further defined as the axis that the end effector rotates around to move side to side relative to the upper and lower portions of the shaft and the pitch axis will be defined as the axis that the end effector rotates around to move up and down relative to the shaft (similar to the pitch and yaw axis of an airplane). However, it will be understood that these definitions can be reversed. The end effector 14 comprises jaw members 14a, 14b configured to pivot relative to each other to move the jaw members 14a, 14b between open and closed configurations (the latter being depicted in FIG. 1).

In certain embodiments, the surgical instruments described herein are adapted to be used with a robotic system for applying ligating clips. The surgical instruments will generally include an actuation mechanism that controls the orientation and movement of the end effector. The actuation mechanism will typically be controlled by a robotic manipulator assembly that is controlled remotely by a user. For example, in one configuration, the actuation mechanism will be manipulated by the robotic manipulator assembly to move the jaws of the end effector between an open position and a closed position. In the closed position, the jaws are actuated into compressing contact with the legs of a clip, thereby compressing the clip into a latched or locked position around a vessel or other tissue.

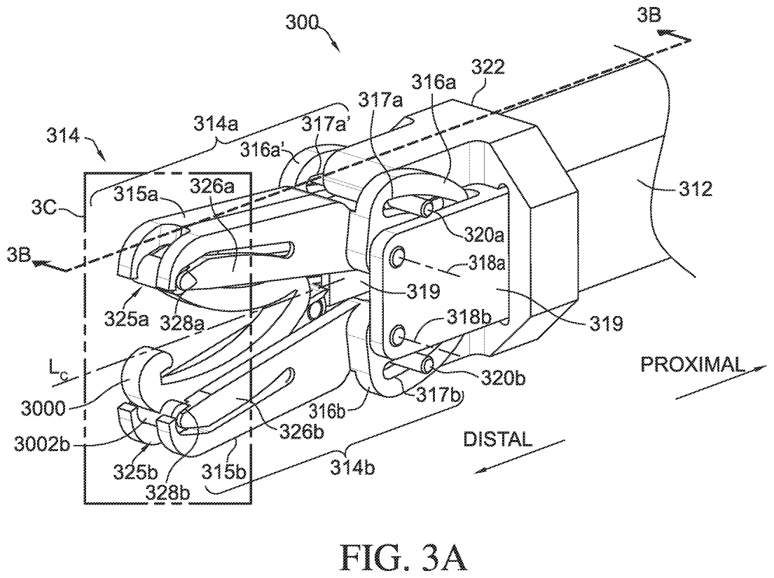

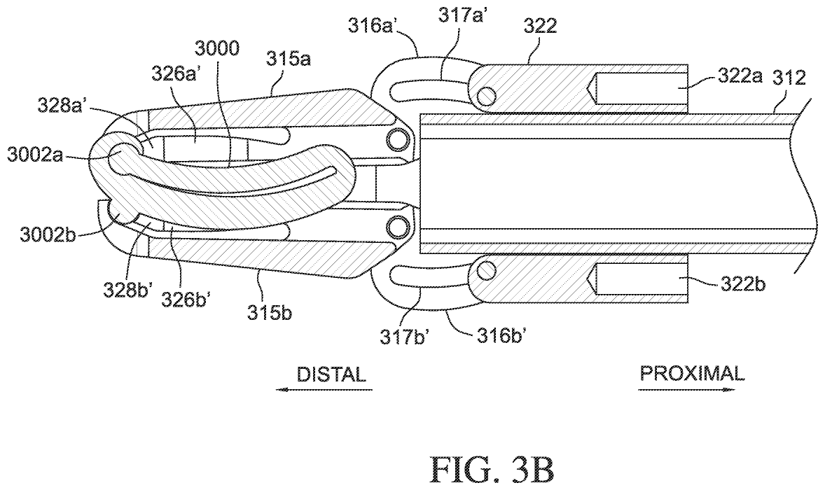

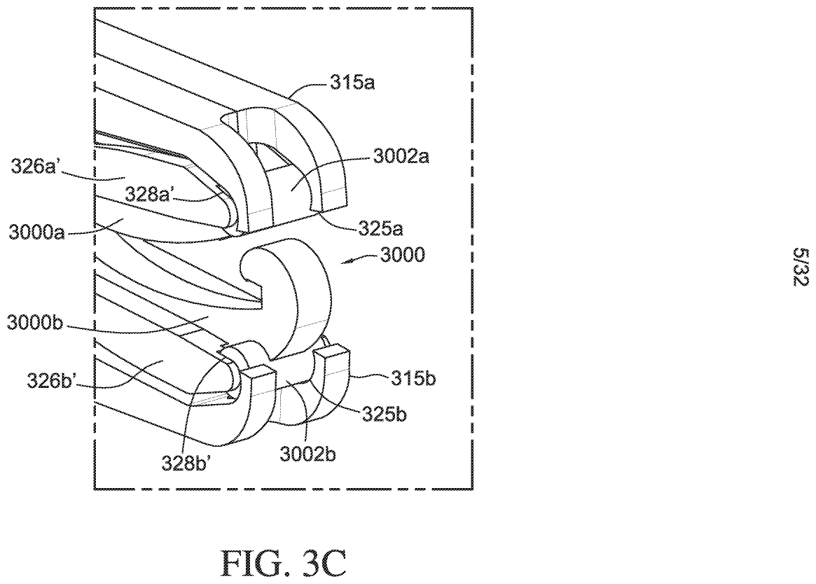

The actuation mechanism may include input couplers (not shown) instead of, or in addition to, the stationary and movable handles. In certain embodiments, surgical instrument 100 will further include a backend mechanism 510 (see FIGS. 3A and 56) coupled to the proximal end portion of elongate shaft 105. The backend mechanism typically provides a mechanical coupling between the drive tendons, rods or cables of the instrument and motorized axes of the mechanical interface of a drive system. Further details of known backend mechanisms and surgical systems are described, for example, in U.S. Pat. Nos. 8,597,280, 7,048,745, and 10,016,244. Each of these patents is hereby incorporated by reference in its entirety.

The input couplers may interface with, and be driven by, corresponding output couplers (not shown) of a telesurgical surgery system, such as the system disclosed in U.S. Pub. No. 2014/0183244A1, the entire disclosure of which is incorporated by reference herein. The input couplers are drivingly coupled with one or more input members (not shown) that are disposed within the instrument shaft 105. The input members are drivingly coupled with the end effector 110. Suitable input couplers can be adapted to mate with various types of motor packs (not shown), such as the stapler-specific motor packs disclosed in U.S. Pat. No. 8,912,746, or the universal motor packs disclosed in U.S. Pat. No. 8,529,582, the disclosures of both of which are incorporated by reference herein in their entirety. Further details of known input couplers and surgical systems are described, for example, in U.S. Pat. No. 8,597,280, U.S. Pat. No. 7,048,745, and U.S. Pat. No. 10,016,244. Each of these patents is hereby incorporated by reference in its entirety for all purposes.

Robotic surgery systems and methods are further described in U.S. Pat. No. 5,797,900, filed on May 16, 1997, issued on Aug. 25, 1998, U.S. Pat. No. 6,132,368, filed on Nov. 21, 1997, issued on Oct. 17, 2000, U.S. Pat. No. 6,331,181, filed on Oct. 15, 1999, issued on Dec. 18, 2001, U.S. Pat. No. 6,441,577, filed on Apr. 3, 2001, issued on Aug. 27, 2002, U.S. Pat. No. 6,902,560, filed on Jan. 6, 2004, issued on Jun. 7, 2005, U.S. Pat. No. 6,936,042, filed on Apr. 16, 2002, issued on Aug. 30, 2005, and U.S. Pat. No. 6,994,703, filed on Dec. 4, 2002, issued on Feb. 7, 2006, the full disclosures of which are incorporated herein by reference for all purposes. A suitable robotic surgical system currently in use is the da Vinci S Surgical System by Intuitive Surgical, Inc.

While described herein with respect to an instrument configured for use with a robotic surgical system, it should be understood that the actuation and drive assemblies described herein may be incorporated into manually actuated instruments, electro-mechanical powered instruments, or instruments actuated in any other way. For example, the actuation mechanism may comprise a handle assembly for gripping by the user that includes a stationary handle and a moveable handle, which serves as an actuator for surgical instrument 100.

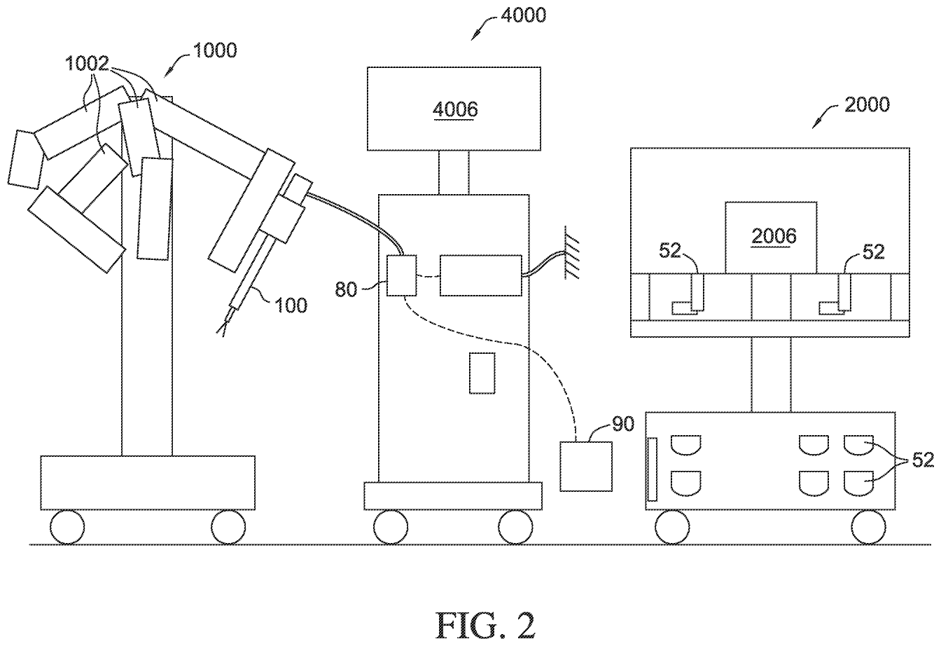

In various embodiments, the force transmission mechanism 10 is configured to operably couple to and receive drive inputs from a manipulator system of a teleoperable medical system that operates at least in part with some computer assistance (sometimes referred to as a robotic surgical system). One embodiment of such a computer-assisted, teleoperable medical system is illustrated in the schematic diagram of FIG. 2, depicting a manipulator system 1000 comprising a plurality of manipulator arms 1002 to which the force transmission mechanism 10 of instrument 100 may operably couple, a surgeon side console 2000 comprising various master inputs 52 and a surgeon viewer 2006 which can include video images of the remote worksite taken through an endoscopic imaging instrument and/or other graphical information, and a vision/control console 4000 which may also have a display 4006 presenting similar images as the viewer 2006 or other information relating to a procedure. The vision/control console 4000 also may in some embodiments comprise components that supply auxiliary functionality to instruments, such as via an auxiliary unit 80, which may be, for example, insufflation gas, vacuum for evacuation, electrosurgical energy, and similar flux supply units. Such units may be controlled through a controller integrated with the system and/or may be separately controlled at a stand-alone input unit 90, rather than through the surgeon console 2000. A non-limiting embodiment of a computer-assisted, teleoperable medical system with which the instrument 100 and various instrument embodiments described herein can be utilized are the da Vinci® Surgical Systems commercialized by Intuitive Surgical, Inc., of Sunnyvale, California.

In various other embodiments, the medical instrument 100 can be configured to be manually actuated, with the proximal end force transmission mechanism 10 having inputs that are configured to be manually actuated rather than coupled to a manipulator arm. Yet other embodiments contemplate the instrument can have both manually actuated inputs and inputs configured to be driven by drive outputs of a manipulator system.

Referring again to FIG. 1, the instrument 100 is configured to be loaded with a plurality of medical clips (not shown), which can be advanced one at a time from a location proximal of the jaw members 14a, 14b of the end effector 14 to between the jaw members 14a, 14b, where an advanced clip can be held by the jaw members 14a, 14b for subsequent application to an object, such as tissue, vessels, or another object. In some embodiments, as mentioned above, the end effector 14 may be coupled to the shaft 12 via a wrist mechanism 13 configured to articulate the end effector 14 in one or both of pitch and yaw relative to the shaft 12. In such embodiments, the jaw members 14a, 14b may pivot relative to one another to move the jaw members 14a, 14b in open and closed states, and they also may also be oriented together in pitch and/or yaw via articulation of the wrist mechanism 13. In cases in which a wrist mechanism 13 couples the end effector 14 to the shaft 12, clips may be advanced through the wrist mechanism to be loaded between grasping portions of the jaw members 14a, 14b.

To enable advancement of one or more clips from a proximal position to be held between the jaw members, various clip guidance and retention features can be used. These features may permit the clips to be advanced in a predictable and accurate manner to a position between the jaw members 14a, 14b, to be held securely between the jaw members 14a, 14b to prevent inadvertent release of a clip prior to desired application and to allow the jaw members to fully open (increase their internal opening angle) to allow the held clip to be positioned as desired over the object to which the clip is to be applied, and to further allow for sufficient force to applied by the jaw members 14a, 14b to move them toward the closed configuration and latch the clip over the desired object.

Referring now to FIGS. 3A-3C, an embodiment of a distal end portion of a medical instrument for applying medical clips that permits advancement of the clips from proximal the end effector and that includes jaw members configured with various clip guidance and retention features is illustrated. The distal end portion of the instrument 300 shown may be an embodiment of the instrument 100, although the instrument 100 is not limited to such embodiment. The clip guidance and retention features of instrument 300, as will be understood further from the following description, are configured to engage with complementary features and arms of a clip. FIG. 3A is a partial perspective view and FIG. 3B is a longitudinal cross-sectional view taken through section 3B-3B in FIG. 3A and showing the jaw members of the instrument in a closed state. FIG. 3C is a detailed view of portion 3C of FIG. 3A.

The instrument 300 comprises a shaft 312 (a portion of which is shown in ghost) to which an end effector 314 comprising jaw members 314a, 314b is coupled. The jaw members 314a, 314b are shown in a partially open state with a medical clip 3000 held between the jaw members 314a, 314b. The jaw members 314a, 314b comprise a pair of opposing grasping portions 315a, 315b that are configured to open and close via relative pivoting motion to receive and hold a clip (clip 3000 in FIG. 3A) and apply force (during closure as shown in FIG. 3B) sufficient to latch the clip 3000 around an object. In various embodiments, the angle between the grasping portions 315a, 315b of the jaw members 314a, 314b in a fully open state ranges from 20 to 45 degrees, for example, from 30 to 35 degrees. Extending proximally from each of the grasping portions 315a, 315b of the jaw members 314a, 314b are connecting portions 316a, 316a′, 316b, 316b′ (shown in FIG. 3B), to which the grasping portions 315a, 315b are respectively pivotably coupled and pivotable about respective rotation axes 318a, 318b. The connecting portions 316a, 316a′, 316b, 316b′ for each jaw member 314a, 314b are positioned opposite a longitudinal centerline Lc of the jaw members 314a, 314b so as to allow for a connection to the shaft 312 that provides space for the clip 3000 to be advanced between the jaw members 314a, 314b from proximally the jaw members 314a, 314b.

The connection portions 316a, 316a′, 316b, 316b′ are pivotably connected to a clevis via opposed clevis extensions 319 that extend in a direction distally away from the shaft 312 and are positioned on opposite sides of the centerline Lc of jaw members 314a, 314b. The clevis can be used to couple the end effector 314 to the shaft 312 or to an optional wrist mechanism (not shown in the embodiment of FIGS. 3A-3B) which may be coupled to the shaft 312. The connection portions 316a, 316a′, 316b, 316b′ are also coupled to an actuation link 322 via pins 320a, 320b as further explained below. Actuation of the jaw members 314a, 314b to cause the pivoting and consequent opening/closing of the grasping portions 315a, 315b occurs through translation of pins 320a, 320b through along slots 317a, 317a′, 317b, 317b′ (shown in FIG. 3B) provided in the connection portions 316a, 316a′, 316b, 316b′. The translation of the pins 320a, 320b is driven by the actuation link 322. The actuation link 322 translates the pins 320a, 320b together distally and proximally to respectively open or close the jaw members 314a, 314b relative to each other via the engagement with the slots 317a, 317a′, 317b, 317b′ and pivoting action of each jaw member 314a, 314b about its respective pivot axes 318a, 318b.

FIG. 3A shows the actuation link 322 and pins 320a, 320b in a position corresponding to the jaw members 314a, 314b being in a partially open state, while FIG. 3B shows the actuation link 322 and pins 320a, 320b, moved proximally in the direction of the arrow shown to place the jaw members 314a, 314b in a fully closed state in which the pins 320a, 320b are at a proximal-most location of the slots 317a, 317a′, 317b, 317b′. A fully open state of the grasping portions 315a, 315b of the jaw members 314a, 314b (not shown) can be achieved by moving the actuation link 322 and pins 320a, 320b, in a distal direction (opposite to the arrow in FIG. 3B) to a location in which the pins 320a, 320b, are at a distal-most location of the slots 317a, 317a′ 317b, 317b′. Although not shown in FIGS. 3A and 3B, it is contemplated that one or more push-pull actuation elements (e.g., rods or other similar members) can be coupled to the actuation link 322 and routed along the instrument shaft 312 for actuation at a proximal transmission mechanism of the instrument. FIG. 3B shows openings 322a, 322b for receipt and coupling of two opposing push-pull actuation elements (not shown). However, any number of such actuation elements (e.g., 1, 2, or more) may be used to create the pushing and pulling forces acting to move the actuation link 322 distally and proximally.

The grasping portions 315a, 315b of the jaw members 314a, 314b have a configuration that enables reliable advancement and retention of the clip 3000 from a position proximal of the grasping portions 315a, 315b (i.e., between the opposed connection portions 316a, 316a′, 316b, 316b′ and the clevis extensions 319). As shown best with reference to grasping portion 315b in FIG. 3A and the detailed view of FIG. 3C corresponding to portion 3C in FIG. 3A (but with FIG. 3C showing the grasping portions 315a, 315b in a partially closed, but not fully closed, state), the grasping portions 315a, 315b, comprise a split configuration that defines a longitudinally extending channel 325a, 325b configured to receive opposing arms 3000a, 3000b of the clip 3000 as it is advanced between the grasping portions 315a, 315b of the jaw members 314a, 314b. Inner surfaces of the portions of the grasping portions 315a, 315b defining the channels 325a, 325b are provided with features defining guidance tracks (see sectional view of FIG. 3B) configured to receive and guide retention bosses 3002a, 3002b, that are provided near or as part of the free, distal latching ends of the arms 3000a, 3000b of the clip 3000. Retention bosses 3002a, 3002b extend oppositely from each side of the arms 3000a, 3000b.

In addition, the grasping portions 315a, 315b comprise leaf springs 326a, 326b, 326a′, 326b′ on either side of the respective channels 325a, 325b. The leaf springs 326a, 326b, 326a′, 326b′ are biased toward the centerline Lc to provide a force to retain the clip arms 3000a, 3000b in the channels 325a, 325b during advancement of the clip 3000. The inner surfaces of the leaf springs 326a, 326b, 326a′, 326b′ further define small, recessed regions 328a, 328b, 328a′, 328b′ on inner end surfaces thereof that are configured to receive and, together with a curved distal end rigid portions of the grasping portions 315a, 315b provide stop surfaces for retaining the bosses 3002a, 3002b of the clip arms 3000a, 3000b from longitudinal movement, particularly distally. Upon closure and latching of the clip 3000 by the jaw members 314a, 314b, the latching force of the latching ends, is sufficient to overcome the retention force of the grasping portions 315a, 315b acting on the clip 3000, allowing it to be released therefrom.

In another embodiment, it is contemplated that a wrist mechanism with sufficient central lumen clearance to permit advancement of clips through the wrist mechanism could be used to couple the actuation link 322 and end effector 314 to the shaft 312 so as to allow articulation of the end effector 314 relative to the shaft 312 in pitch and/or yaw.

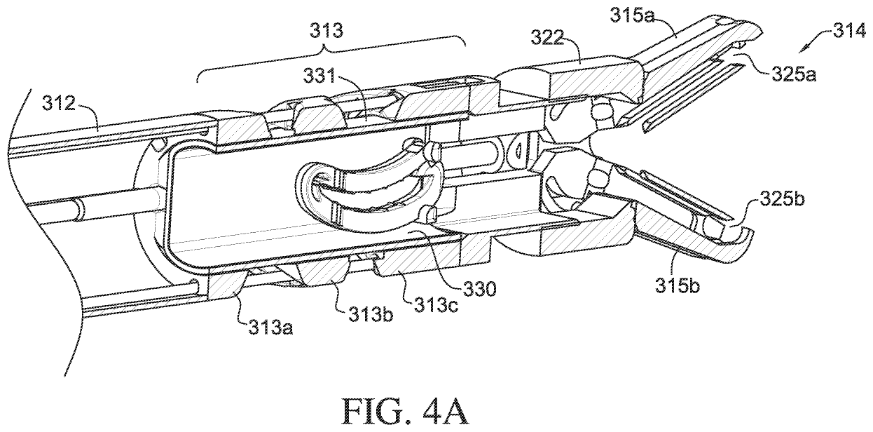

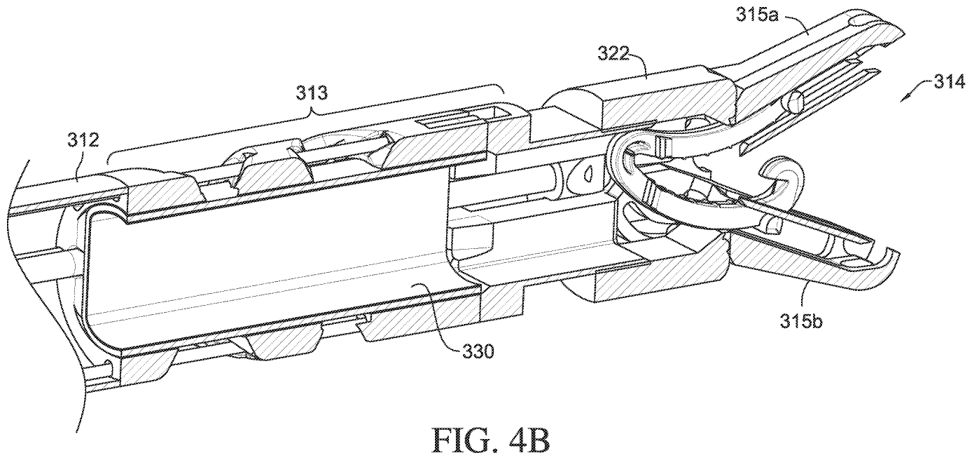

One embodiment of a wrist mechanism that can be used to couple the actuation link 322 and end effector 314 to the instrument shaft 312 is depicted in FIGS. 4A and 4B. The wrist mechanism 313 shown comprises a plurality of links 313a-313c (3 being shown but 2 or more may be used), coupled in series via joints between adjacent links. Actuation members, such as cables or the like, that can be paid in and paid out can be coupled to cause the forces uses to cause the links to articulate about the joints so as to thus cause the end effector 314 to be oriented relative to the shaft 312. Those having ordinary skill in the art are generally familiar with this type of wrist mechanism. By using links 313a-313c having an annular shape with a relatively large central opening such that when coupled in series a relatively large central lumen 330 is created, the wrist mechanism 313 can allow for advancement of a medical clip 3000 through the central lumen 330, as depicted in FIG. 4A, and into the channels 325a, 325b of the grasping portions 315a, 315b of the jaw members 314a, 314b, as described above and shown in FIG. 4B.

To avoid the clip 3000 catching on the links 313a-313c of the wrist mechanism 313 during advancement, a flexible sleeve 331 or other similar guidance structure, such as a flexible ribbon structure described with respect to various embodiments further below, can be placed in the central lumen 330. The sleeve 331, or other guidance structure, can be configured so as to elastically flex when the wrist mechanism 313 articulates while providing a relatively smooth inner surfaces through which the clip 3000 can pass during advancement through the wrist mechanism 313 while retaining a position relative to the lumen allowing it to provide the smooth surface between the clip and the wrist mechanism 313.

As further illustrated in FIGS. 4A and 4B, the clip 3000 can be advanced in a closed configuration through the wrist mechanism 313, allowing for a relatively small overall dimensions of the wrist mechanism 313, and then may spring to an open configuration once the clip 3000 is advanced into at least partially open grasping portions 315a, 315b of the jaw members 314a, 314b. In other words, the clip 3000 may be elastically biased toward an open configuration (or at least a partially open configuration), but be held in a closed configuration as it advances through the wrist mechanism 313. Upon external forces acting to close the clip 3000, the clip arms 3000a, 3000b may then spring to an at least partially open configuration. It is further contemplated that once the clip arms 3000a, 3000b are further retained by the respective grasping portions 315a, 315b of the jaw members 314a, 314b, the clip 3000 can be further opened without elastic deformation by further opening the grasping portions 315a, 315b, which may be useful to allow placement of the clip arms 3000a, 3000b around relatively large objects to be clipped.

Another embodiment of a wrist mechanism that is articulatable in at least one degree of freedom, such as, for example, pitch (defined herein as about an axis perpendicular to the longitudinal axis of the shaft and to the pivot axes of the jaw members) that provides sufficient room to permit advancement of medical clips (one at a time sequentially) through the wrist mechanism relies on a bar linkage. The bar linkage provides a channel through an interior of the linkage that provides a path for the advancement of clips in both a neutral position of the wrist and through a range of articulation of the wrist from the neutral position. The bar linkage can also permit other pulley and cable actuation mechanisms to control other movement of the end effectors and/or additional wrist joints, without the actuation of such additional movement affecting the articulation of the pitch wrist mechanism and vice versa.

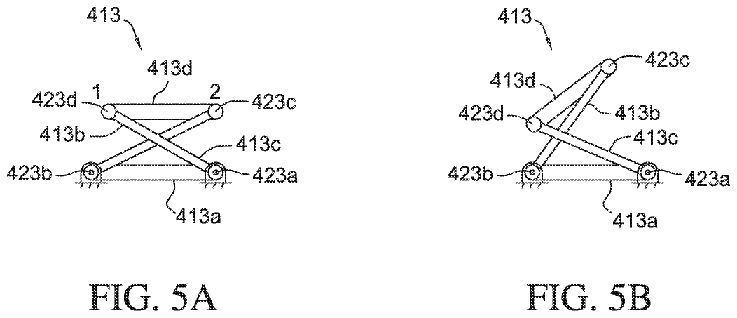

With reference to FIGS. 5A and 5B, a schematic illustration of an embodiment of a crossed four-bar linkage that can be implemented for use in a wrist mechanism to connect an end effector for applying clips to a medical instrument shaft, such as shaft 312 and end effector 314 of the embodiment of FIGS. 3A-3C, is illustrated. The crossed four-bar linkage wrist mechanism 413 permits articulation through a range of motion in a single degree of freedom (e.g., pitch relative to the shaft and pivot axis of the jaw members of the end effector, as will be explained further below). The crossed four-bar linkage generally comprises a fixed link 413a (which can be the link fixedly coupled to the shaft or a part of the instrument shaft itself, such as instrument shaft 312), two bar links 413b, 413c, and another moveable link 413d (which can be fixed to the clevis of the end effector or can be part of the clevis itself, such as the end of the clevis opposite clevis extensions 319).

The bar links 413b, 413c pivotably couple to the fixed link 413a at diametrically opposite positions relative to a longitudinal centerline of the linkage and cross each other as they extend across the longitudinal centerline of the linkage from the fixed link 413a to the moveable link 413d, where they are again pivotably coupled so as to be diametrically opposite each other. The opposite ends of each of the bar links 413b, 413c are pivotably coupled at fixed pivot couplings 423a, 423b to the fixed link 413a and moveable pivot couplings 423c, 423d (e.g., in translation in response to pivoting about the fixed pivot locations) to the moveable link 413d, respectively, and are also coupled on opposite sides of the longitudinal centerline. Because the bar links 413b, 413c can couple to the fixed and moveable links (which can be generally annular in shape in the context of an instrument) toward outer peripheries of those links, the movement of the bar links 413b, 413c about their pivot couplings can permit sufficient space between the links 413b, 413c to provide a path for advancement of a clip through the four-bar linkage wrist mechanism 413. Moreover, this path can be maintained throughout a range of motion of articulation of the four-bar linkage wrist mechanism 413. For example, the path can be maintained through a range of motion from neutral (zero degrees of relative to the longitudinal axis of the fixed link 413a, e.g., instrument shaft) through at least +/−45 degrees. In some embodiments, for example the range of motion from neutral may be through +/−60 degrees, or for example, through +/−65 degrees.

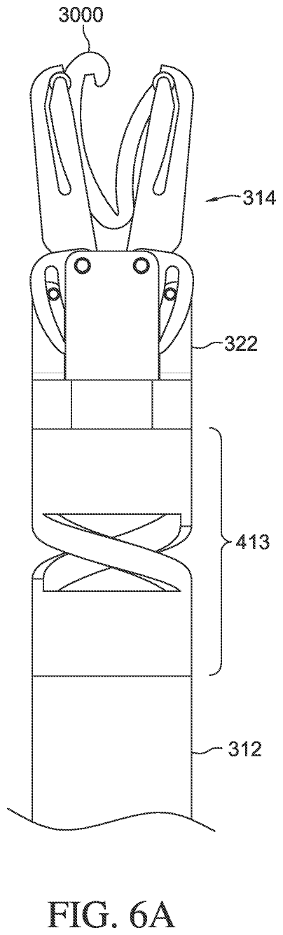

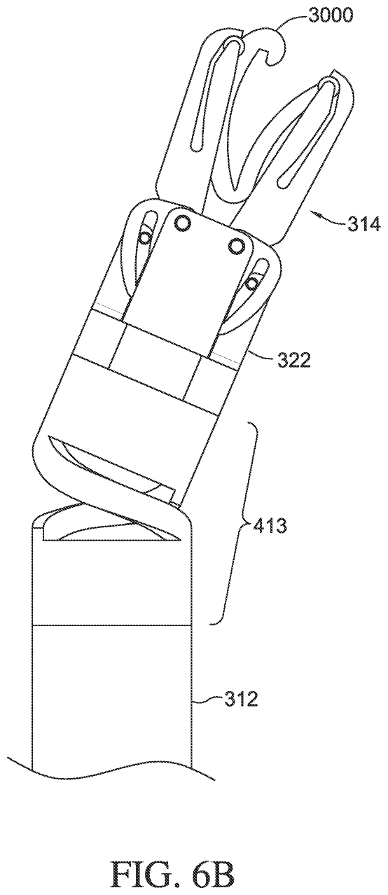

Referring now to FIGS. 6A and 6B, an embodiment of the end effector 314 and actuation link 320 of FIGS. 3A-3C shown coupled to an instrument shaft 312 using a crossed four-bar linkage wrist mechanism 413 so as to provide pitch articulation of the end effector relative to the shaft is illustrated. FIG. 6A shows the wrist mechanism 413 in a neutral state in which the longitudinal axis of the wrist mechanism 413 is aligned with the longitudinal axis of the shaft 312, and FIG. 6B shows the wrist mechanism 413 articulated relative to the shaft 312 (e.g., in pitch). One or more pairs of opposing actuation members such as cables or the like (not shown), can be used to generate the force to articulate the wrist mechanism as would be understood by those having ordinary skill in the art, with the actuation members (not shown) being paid in and paid out depending on the direction of articulation relative to the shaft 312. The crossed four-bar linkage can provide a sufficiently open central region to allow for a medical clip to be advanced therethrough throughout a range of articulation of the wrist mechanism 413. The crossed four-bar linkage wrist mechanism 413 also can allow for the actuation members to be routed toward an outer periphery (either externally or internally of the wrist mechanism) so that they do not take up the central region of the wrist mechanism 413 where it is desirable to advance the clip 3000, thus enabling relatively small overall lateral (e.g., radial) dimensions of the wrist mechanism.

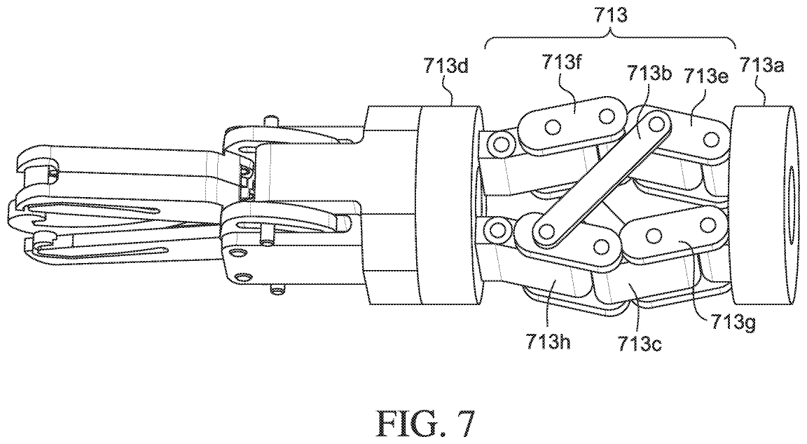

Other configurations of bar-type linkages may be used to couple an end effector of a clip applier to an instrument shaft and that permit sufficient space to advance a clip therethrough so as to enable clip advancement from proximal of the end effector in accordance with the present disclosure. For example, as depicted in the schematic view of FIG. 7, an eight-bar cross-linkage may be used as the wrist mechanism 713 to attach between an instrument shaft (depicted schematically by link 713a) and a clevis portion of the end effector (depicted by link 713d). The remaining six links may be bar links (713b, 713c, 713e, 713f, 713g, 713h) pivotably coupled to each other and the links 713a, 713d, as shown. Such a linkage may provide more space for advancing clips, while also providing a structural passage for clips that minimizes gaps and other regions that may catch a clip as it advances through the wrist mechanism, even in an articulated state of the wrist mechanism.

While the end effectors of FIGS. 4-7 depict the jaw members and actuation link of the embodiment of FIGS. 3A-3C, such a configuration is nonlimiting and other embodiments of end effectors for applying clips may be used in conjunction with the wrist mechanisms of FIGS. 4-7, including various end effector embodiments further shown and described below.

The present disclosure further contemplates embodiments in which a wrist mechanism is configured to articulate so as to orient the end effector (e.g., jaw members) relative to the shaft of the instrument in pitch or yaw depending on the orientation of the device relative to the user (defined herein as about an axis perpendicular to the longitudinal axis of the shaft and parallel to the pivot axis about which the jaw members open/close) and that provides sufficient room to permit advancement of medical clips (one at a time sequentially) through the wrist mechanism. Such embodiments also permit sufficient clip closure force throughout various ranges of articulation of the wrist mechanism and also may have various guidance and retention features to allow for clip advancement from proximally to the jaw members.

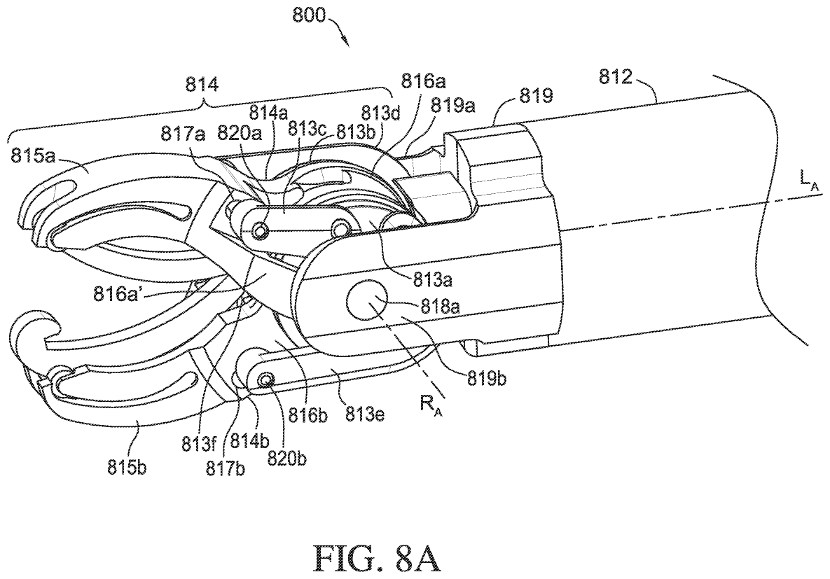

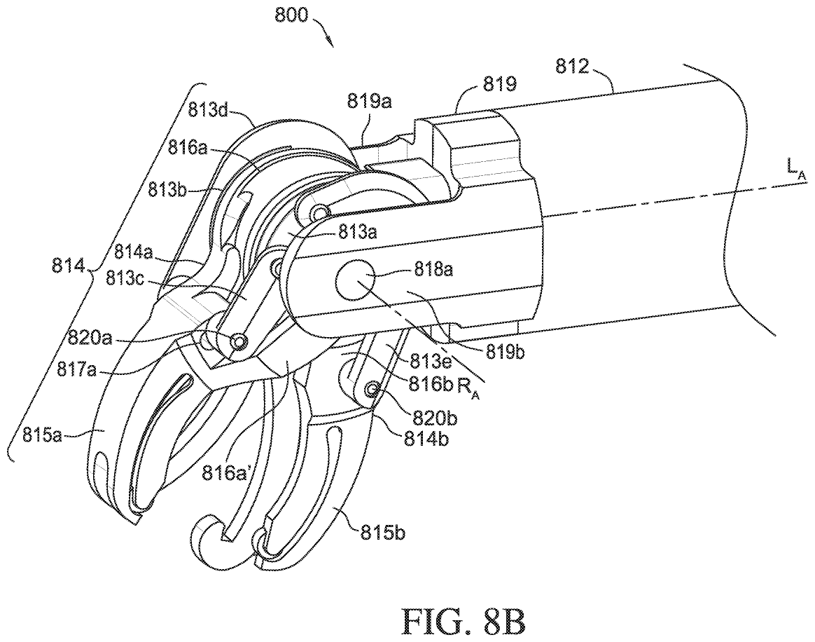

With reference to FIGS. 8A-10D, an embodiment of a distal end portion of a medical instrument 800 for applying medical clips that incorporates various guidance and retention features for the clips, yaw or pitch articulation via a wrist mechanism, sufficient closure force to apply a medical clip, and enables clip advancement from proximal the end effector (so as to permit multi-clip applications) will now be described. The distal end portion of the embodiments of FIGS. 8A-10D can be used as the distal end portion of the instrument 100, but the instrument 100 may encompass other distal end portions. The instrument 800 utilizes a pulley and linkage system that provides relative pivoting of the jaw members (for opening and closing) and a pitch or yaw articulation wrist mechanism about the same axis. Use of the same, single pivot axis helps to minimize gaps that can form between sections of the overall link structure (wrist links and end effector links) which can pose difficulties for smoothly and reliably advancing clips. For example, gaps may lead to a portion of a clip catching and getting stuck during advancement. The single pivot axis can maintain a more compact longitudinal configuration of the end effector and wrist mechanism with the pulley and linkage system architecture of the embodiment of FIGS. 8A-10D providing a fixed location of the pivot axis of the jaw members relative to the wrist mechanism along the longitudinal axis.

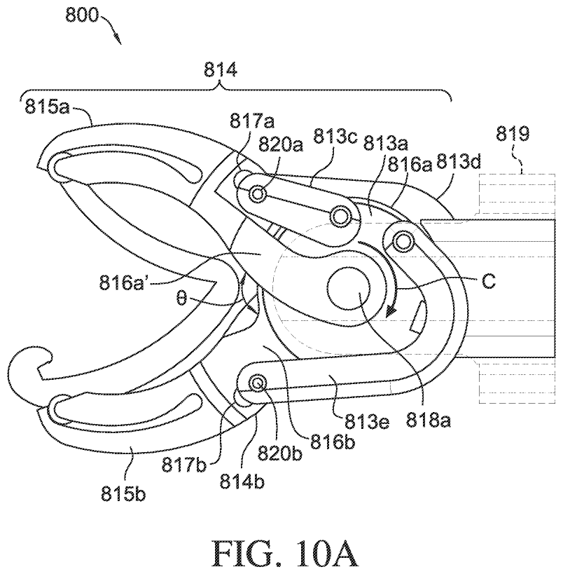

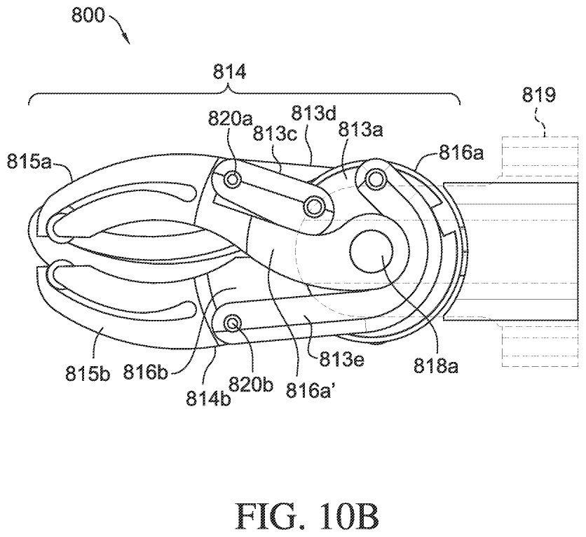

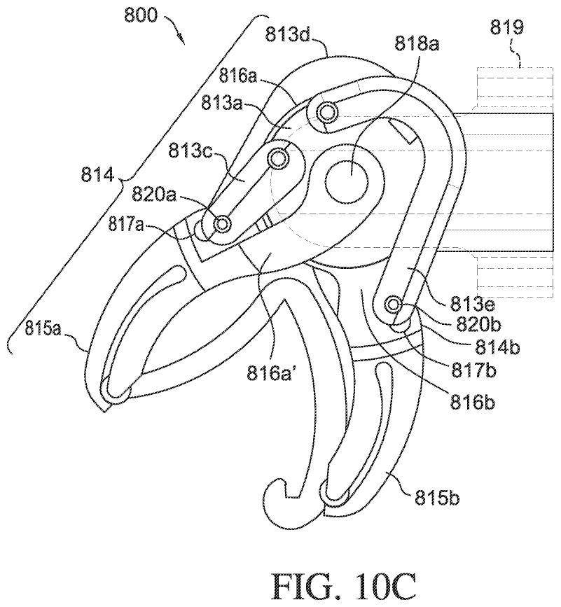

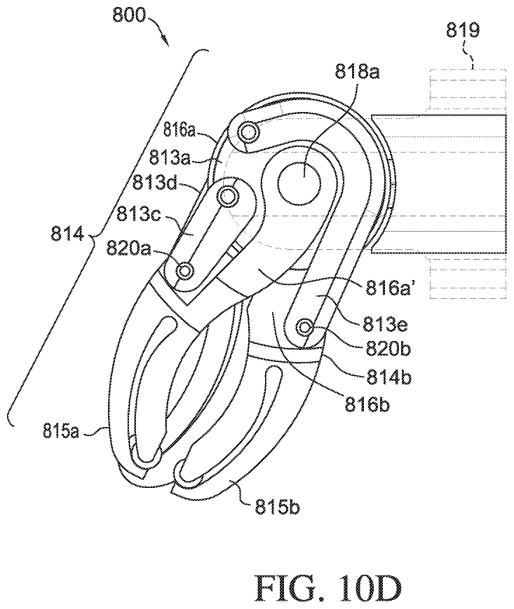

FIGS. 8A-10D show the pulley and linkage system that provides for both a wrist mechanism that articulates the end effector in yaw or pitch (jaw members together in yaw or pitch) relative to the instrument shaft and that also pivots the jaw members relative to each other about the yaw or pitch articulation axis so as to open and close the jaw members. FIGS. 8A and 8B are perspective side views of the end effector 814 with connection portions 816a, 816a′, 816b, 816b′ of the opposing jaw members 814a, 814b attached to the clevis 819 via clevis extensions 819a, 819b, with FIG. 8A showing the wrist mechanism 813 in a neutral configuration (unarticulated relative to the longitudinal axis L_{A} of the shaft 812) and FIG. 8B showing the wrist mechanism 813 in a state fully articulated in yaw or pitch relative to the longitudinal axis L_{A} of the shaft 812. The clevis extensions 819a, 819b are shown transparent in FIGS. 10A-10D to provide a better view of internal components and attachments of the end effector 814 and wrist mechanism 813.

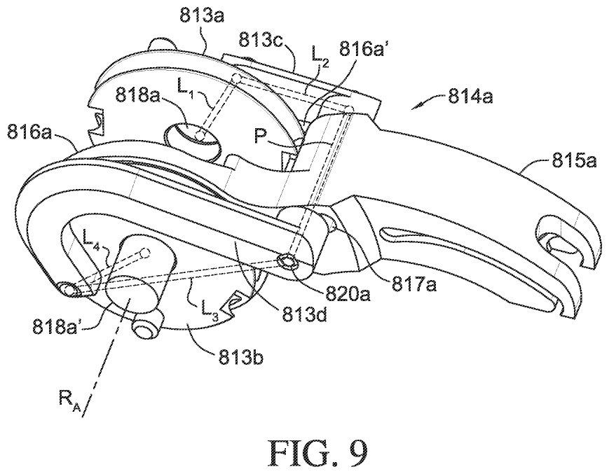

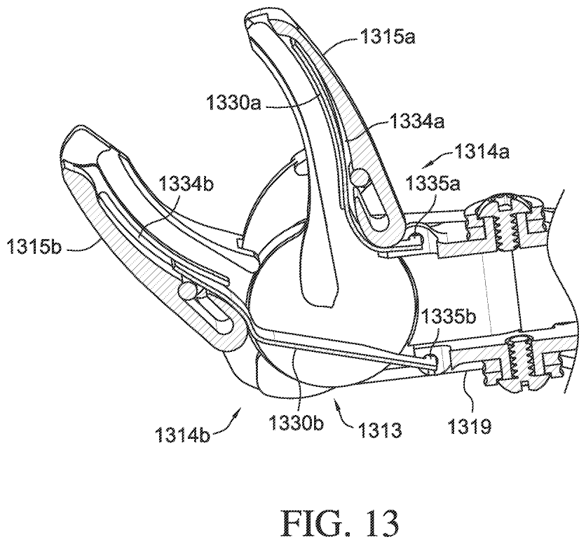

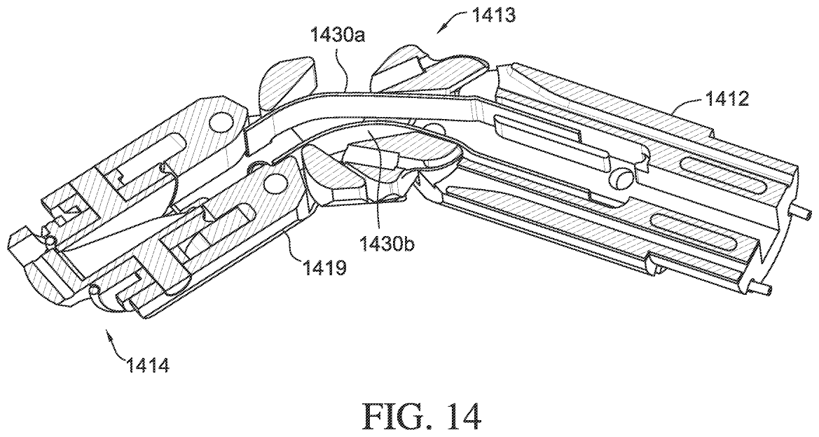

In the embodiment of FIGS. 8A-10D, the clevis 819 couples the end effector 814 to the shaft 812, but in other embodiments it is contemplated that an additional wrist mechanism could be used to couple the clevis 819 to the shaft 812, such as, for example as described further below with reference to the embodiment of FIGS. 13-14. FIG. 9 is a perspective, isolated view of jaw member 814a and the pulley and linkage system for pivoting of the grasping portion 815a of jaw member 814a relative to the grasping portion 815b of jaw member 814b to open/close the grasping portions 815a, 815b and for use as a wrist mechanism 813 for articulation of the jaw member 814a in yaw or pitch relative to the shaft 812. The single jaw member 814a is illustrated to better show and describe various components and details. The opposing jaw member 814b is connected to the pulleys shown in FIG. 9 via oppositely disposed and connected links, as can be seen in FIGS. 8A, 8B and 10A-10D and will be further understood from the following description.

The pulley and linkage system that provides the wrist mechanism 813 to provide yaw or pitch articulation and the relative pivoting open/close degree of freedom of the grasping portions 815a, 815b of the jaw members 814a, 814b comprises a pair of pulleys 813a, 813b facing each other and disposed on opposite sides of the longitudinal axis L_{A} of the shaft 812 and clevis 819 in a neutral yaw or pitch articulation of the wrist mechanism 813 and thus jaw members 814a, 814b. The pulleys 813a, 813b are rotatable about rotation axes R_{A} in both directions in response to actuation of cables (not illustrated) wrapped around each pulley 813a, 813b. Each pulley 813a, 813b is also rotatably coupled to the connection portions 816a, 816a′, 816b, 816b′ of the respective jaw members 814a, 814b, with connection portion 816a being coupled inside pulley 813b via a pin 818a and connection portion 816a′ being coupled outside pulley 813a via a pin 818a′, as perhaps best illustrated in FIG. 9. Jaw member 814b is similarly, but oppositely connect to each pulley 813a, 813b. That is connection portion 816b is rotatably coupled inside pulley 813a via a pin (not visible in FIGS. 8A-9) extending from connection portion 816b and connection portion 816b′ is rotatably coupled outside pulley 813b via a pin (not visible in FIG. 8A-9). The pins also provide the coupling to the clevis extensions 819a, 819b. Reference is also made to FIGS. 10A-10D which show the clevis extensions 819a, 819b partially transparent.

Each jaw member 814a, 814b also connects to the pulleys 813a, 813b via a pair of links comprising a short link 813c, 813f (short link connecting jaw member 814b to pulley 813b barely visible in the view of FIG. 8A) and a long link 813d, 813e. More specifically, the jaw member 814a connects to pulley 813a via the short link 813c and connects to pulley 813b via long link 813d. Jaw member 814b connects to pulley 813a via the long link 813e and the other short link 813f. The short and long links are each pivotably coupled to the respective pulleys 813a, 813b on outer surfaces thereof. As can be seen in FIGS. 8A-9, with the pair of links 813c and 813d being representative, the pair of links extend from jaw member 814a above (in the orientation of the figures) the rotational axis R_{A} of the pulleys 813a, 813b, while the pair of links 813e, 813f extend from jaw member 814b below (in the orientation of the figures) the rotational axis R_{A}. The end portions of the long links 813d, 813e that couple to the pulleys 813a, 813b are curved so that they can be fixedly coupled to the pulleys 813a, 813b at locations aligned with or on an opposite side of the rotational axis R_{A} from which the long links 813d, 813e initially respectively extend to the pulleys 813a, 813b from the jaw members 814a, 814b.

Each pair of links (i.e., short and long links in each pair) also respectively rotatably couples to each jaw member 814a, 814b at opposite end portions of the links from the end portions that connect to the pulleys 813a, 813b. Referring to FIG. 9 as representative, distal end portions of each of the short link 813c and the long link 813d are coupled to an intermediate region of the jaw member 814a located between the grasping portion 815a and the connection portions 816a, 816a′. The links 813c, 813d are coupled via a pin 820a received in a slot 817a of the intermediate region of the jaw member 814a. The slot 817a is sized to allow a small amount of translation of the pin 820a (proximally and distally) relative to the slot 817a. The coupling of the links 813c, 813d to the jaw member 814a via the pin 820a defines a pivot axis about which jaw member 814a can pivot, as will be explained further below. With reference to FIGS. 8A and 8B, jaw member 814b is similarly coupled to the distal end portions of the long link 813e and the short link 813f via a pin 820b received in a slot 817b of an intermediate region of jaw member 814b that is proximal to the grasping portion 815b and distal to the connection portions 816b, 816b′ of the jaw member 814b. A pivot axis for the jaw member 814b is thus defined which is parallel to pivot axis in the neutral articulation state of the wrist mechanism 813. Slot 817b, like slot 817a, also has a sufficient size to allow a small amount of translation of the pin 820b (proximally and distally).

Accordingly, as shown in FIG. 9, the connections of the pair of links (long and short links) and pulleys to a jaw member (jaw member 814a shown in FIG. 9) provides a four-bar linkage system, with pulley 813a acting as a first link (depicted schematically as L1), pivotally connected to short link 813c, which acts as a second link (depicted schematically as L2). The long link 813d acts as a third link (depicted schematically as L3), which is pivotally connected to the second link L2 via the pint 820a and to the pulley 813b, which acts as a fourth link (depicted schematically as L4). A similar four-bar linkage system connects to jaw member 814b, but with the two of the links being provided by the long link 813e and short link 813f instead of links 813c, 813d.

As will be further explained below with reference to FIGS. 10A-10D, the overall pulley and linkage system created by the pulleys 813a, 813b, and pairs of short and long links 813c-813f provides for both the pivoting of the grasping portions 815a, 815b of the jaw members 814a, 814b relative to each other in an open/close degree of freedom and also articulation of the wrist mechanism 813 in yaw or pitch to orient the jaw members 814a, 814b about the shaft longitudinal axis L_{A}. The pulley and linkage system also provides sufficient interior space to allow advancement of a clip between the pulleys 813a, 813b and connection portions 816a, 816a′, 816b, 816b′ and thus from proximal the jaw members 814a, 814b. In addition, positioning of the coupling of the short links close to the where the jaw members 814a, 814b scissor (regions overlap each other) as they close, amplifies the closure force acting on the clip, in a manner similar to a vice grip.

With reference to FIGS. 10A and 10B, which are side views of the end effector 814 with portions shown transparent to better view certain components, to actuate pivoting of the jaw members 814a, 814b relative to each other to effect opening and closing of the grasping portions 815a, 815b, the pulleys 813a, 813b are rotated in opposite directions. Rotation of pulleys 813a, 813b in opposite directions pivots jaw member 814a, 814b throughout a range of motion between an open position of the grasping portions 815a, 815b (shown in FIG. 10A) and a closed position of the grasping portions 815a, 815b (shown in FIG. 10B). In various embodiments, the grasping portions 815a, 815b may open to an included angle ranging from 20 degrees to 45 degrees between the grasping portions 815a, 815b, for example from 30 degrees to 35 degrees.

More specifically, as shown in FIG. 10A, rotation of pulley 813a clockwise (as shown by the arrow C in the view shown in FIG. 10A), while rotating pulley 813b in the opposite direction (counterclockwise), causes jaw members 814a, 814b to pivot away from each other toward the open position. As can be seen in FIG. 10A, in this configuration the pins 820a, 820b move proximally in slots 817a, 817b, while jaw member 814a rotates about rotation axis R_{A} (also in the clockwise direction) and jaw member 814b rotates about rotation axis R_{A} (in the counterclockwise direction). In response to rotation of the pulley 813a, 813b, short link 813c and long links 813d also rotate about their respective pivot couplings to the pulleys 813a, 813b. Similarly, rotation of pulley 813b counterclockwise causes the short link 813f connecting to jaw member 314b (not shown in FIGS. 10A and 10B) and the long link 813e to pivot about their respective pivot couplings to the pulleys 813a, 813b. To close the jaw members 814a, 814b, as shown in FIGS. 10A and 10B, the rotations described above are reversed, with pulley 813a being rotated counterclockwise (CC shown in FIG. 10B) and pulley 813b being rotated clockwise, in turn causing the reverse pivoting actions of the jaw members 814a, 814b and various links.

FIGS. 10C and 10D illustrate similar views of the end effector 814 as in FIGS. 10A and 10B, except showing the wrist mechanism 813 articulating the jaw members 814a, 814b articulated in yaw or pitch relative to the longitudinal axis L_{A} of the instrument shaft. To articulate the jaw members 814a, 814b in yaw or pitch, both pulleys 813a, 813b are rotated in the same direction. For example, rotating both pulleys 813a, 813b clockwise about the rotational axis R_{A} articulates the jaw members 814a, 814b together in first yaw or pitch direction (such as depicted in FIGS. 10C and 10D) and rotating both pulleys 813a, 813b counterclockwise about the rotational axis R_{A} articulates the jaw members 814a, 814b together in yaw or pitch in a second direction opposite to the first direction, or vice versa. By rotating both pulleys 813a, 813b together, the relative positioning of the pivot axes of the various pivot couplings (i.e., links to pulleys and to jaw members) is maintained, but their individual positions rotated through the angular range of motion of the rotation of the pulleys 813a, 813b. Thus, once a relative positioning of the jaw members 814a, 814b to open/close the grasping portions 815a, 815b has been set via rotation of the pulleys 813a, 813b in opposite directions, further rotation of the pulleys 813a, 813b together will produce yaw or pitch articulation of the wrist mechanism 813 and thus the jaw members together.

It also is possible to achieve pivoting of one jaw member relative to the other while holding the other jaw member stationary (i.e., opening and closing a single jaw member relative to the other) by controlling a ratio at which one of the pulleys is rotated relative to the other. In addition, once the jaw members 814a, 814b have been articulated yaw or pitch by rotation of both pulleys 813a, 813b in the same direction, further differential rotation of the pulleys 813a, 813b, can actuate the pivoting of the jaw members 814a, 814b to open/close the same, as described above with regard to FIGS. 10A and 10B.

Because the space between the connection portions 816a, 816b remains constant throughout the range of both yaw or pitch articulation and the relative pivoting of the jaw members 814a, 814b to open and close the grasping portions 815a, 815b, and due to the relatively large portion of the perimeter of the connection portions 816a, 816b and pulleys 813a, 813b providing access to that space, the ability to advance a medical clip from proximal the wrist mechanism 813 (e.g., from the clevis 819) remains feasible throughout a relatively large range of motion of yaw or pitch articulation in both directions from the neutral position. In various embodiments, the range of motion of articulation about the rotational axis R_{A} to allow for yaw from the neutral position ranges is through at least +/−45°, such as for example through +/−60°, or for example through +/−65°. Moreover, and as mentioned above, the configuration of the pivoting of the jaw members 814a, 814b for the open/closing degree of freedom motion and the articulation of the jaw members 814a, 814b in yaw or pitch about the same axis minimizes gaps, e.g., between the wrist mechanism 813 and the jaw members 814a, 814b along the direction of advancement of a clip between the jaw members 814a, 814b, so as to avoid or prevent the risk of the clip being caught in a gap during advancement.

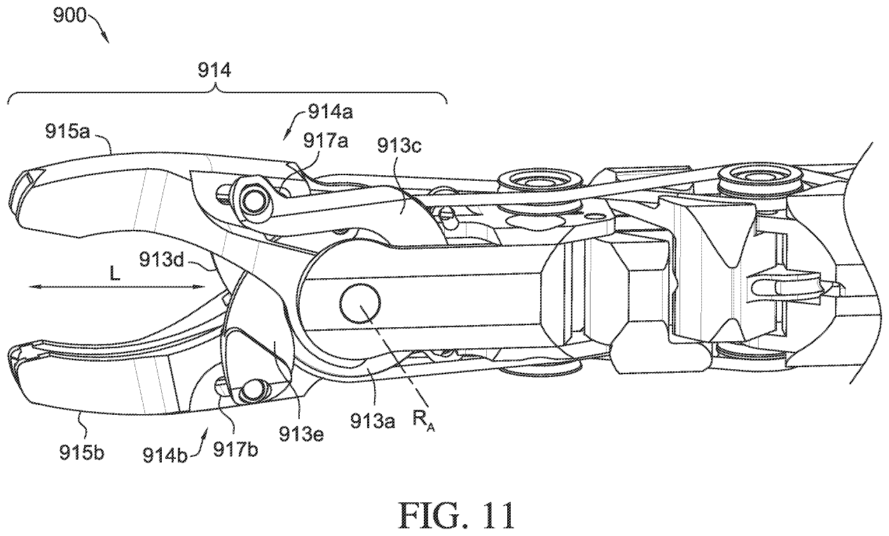

The arrangement of the pulley and linkage system that provides the yaw or pitch articulation and opening/closing of the jaw members of FIGS. 8A-10D is one embodiment of such a pulley and linkage system. FIG. 11 illustrates another embodiment of an end effector 914 of a medical instrument 900 (the distal end portion of which is depicted in the various views) for applying medical clips which has similar parts as the embodiment of FIGS. 8A-10D but with a different configuration of the pulley and linkage system wrist mechanism used for yaw or pitch articulation and opening/closing of the grasping portions of the jaw members. As with other embodiments described herein, the embodiment of FIG. 11 also provides sufficient closure force to apply a medical clip and enables clip advancement from proximal the end effector (e.g., so as to permit multi-clip applications). To avoid redundancy, parts and operation that are in common with the embodiment of FIGS. 8A-10D are not described again here.

The end effector 914 utilizes a pulley and linkage system that provides relative pivoting of the jaw members 914a, 914b (for opening and closing the grasping portions 915a, 915b relative to each other) and a yaw or pitch articulatable wrist mechanism 913 about the same axis. As discussed above, use of the same, single pivot axis helps to minimize gaps that can form between sections of the overall link structure (wrist links and end effector links) which can pose difficulties for smoothly and reliably advancing clips. For example, gaps may lead to a portion of a clip catching and getting stuck during advancement. The single pivot axis can maintain a more compact longitudinal configuration of the end effector and wrist mechanism with the pulley and linkage system architecture of the embodiment of FIG. 11, like that of FIGS. 8A-10D, providing a fixed location of the pivot axis of the jaw members relative to the wrist mechanism along the longitudinal axis.

In the embodiment of FIG. 11, the pairs of links that respectively connect the jaw members 914a, 914b to the respective pulleys 913 (only 913a visible in the view of FIG. 11) have a different arrangement and configuration than the respective pairs of links (e.g., 813c, 813d) connecting the jaw members 814a, 814b to the pulleys 813a, 813b. As can be seen, one of the links 913c being depicted has a similar configuration as the long links (813d, 813e in the embodiment of FIGS. 8-10D) that allows the link to attach to the slots 817a, 817b of the jaw members 814a, 814b on one side and attach to the respective pulleys 913a (other pulley not shown in FIG. 11) at a connection location at a relatively proximal side of the axis of rotation R_{A}. That is, the link 913c shown can be considered a long link it its connection to the pulley is more proximal than the other link 913d of its pair (barely visible in FIG. 11 and which can be considered a short link) and has a curvature allowing it to bend around the axis of rotation R_{A} to connect to the respective pulley (913a shown), and thus spans a slightly longer distance along the longitudinal direction (L in FIG. 11) between where it connects to a respective jaw member and where it connects to a respective pulley. The other link 913d, 913e of the respective pairs of links connecting each jaw member 914a, 914b to the respective pulleys (913a shown), connects from the slot 917a, 917b of each jaw member 914a, 914b and extends around a distal side of the axis of rotation R_{A} to connect to the respective pulleys (connection of link 913e between jaw member 914b and pulley 913a shown) at a location somewhat distal along the longitudinal direction L to where the connection of the link 913c is in the state of the jaw members 914a, 914b shown in FIG. 11 (i.e., partially open and neutral with respect to yaw or pitch articulation).

As with the embodiment of FIGS. 8A-10D, the respective connections of the pairs of links to each jaw 914a, 914b and each pulley 913a, 913b, is the mirror opposite about the longitudinal axis of the end effector 914. The operation of the pulley and linkage system of the embodiment of FIG. 11 to articulate the jaw members 914a, 914b in yaw or pitch and to pivot the jaw members 914a, 914b to open/close the grasping portions 915a, 915b is the same as that described above with respect to the embodiment of FIGS. 8A-10D. However, the arrangement of the pulley and linkage system of FIG. 11 may provide a more uniform mechanical advantage for closure of the grasping portions 915a, 915b of the jaw members 914a, 914b throughout the range of motion, thus enhancing responsiveness to actuation forces applied to closure of the jaw members. Ranges of motion for open/closing the grasping portions of the jaw members and yaw or pitch articulation are the same as those for other embodiments described herein.

To help enable reliable advancement of a medical clip through the clevis and into the grasping portions of the jaw members in an embodiment which uses the type of pulley and linkage system for jaw member open/closing motion and wrist mechanism as described with reference to the embodiments of FIGS. 8A-11, various embodiments may rely on one or more clip guidance and retention features, some of which may be similar in structure and design as those described with reference to the embodiments of FIGS. 3A and 3B described above.

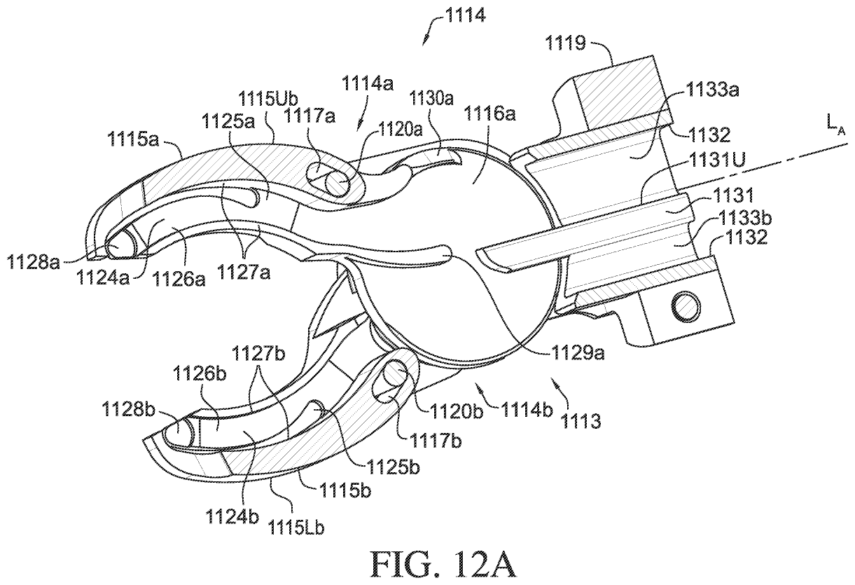

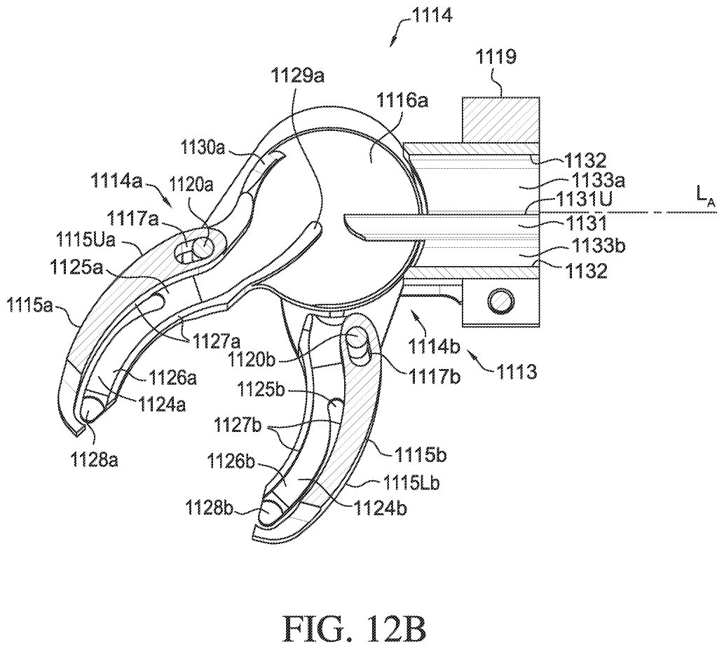

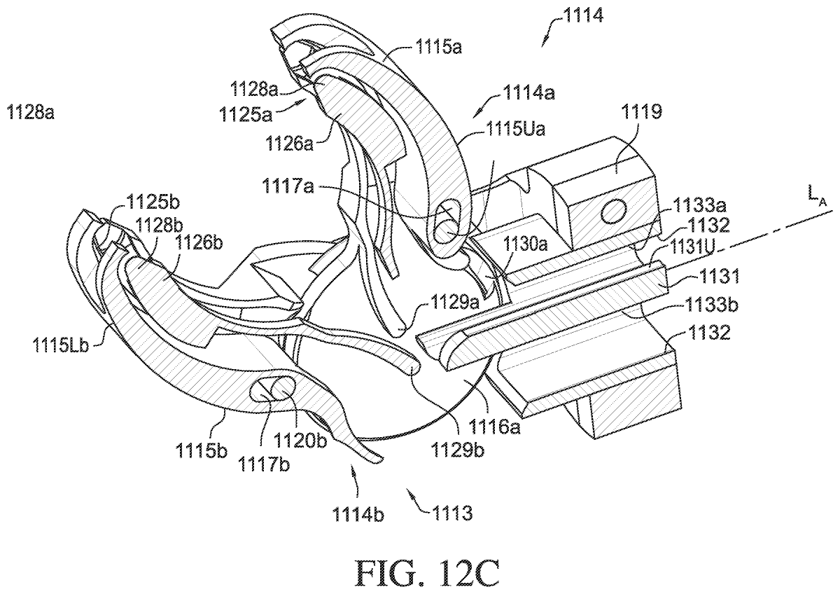

Referring now to FIGS. 12A-12C, longitudinal cutaway views of an embodiment of an end effector 1114 comprising similar structural components as the end effector 814 is depicted, with similar parts being labeled with reference numerals having the same last two digits following an 11xx series rather than the 8xx series. FIGS. 12A and 12B are views taken in the same longitudinal plane cutting generally along a longitudinal centerline of the end effector, while FIG. 12C is a view taken in a different longitudinal plane that is radially outward from the longitudinal centerline toward the plane of the drawing sheet. FIG. 12A shows the grasping portions 1115a, 1115b of the jaw members 1114a, 1114b of the end effector 1114 in an open position and a neutral state of the wrist mechanism 1113 (unarticulated relative to the longitudinal axis L_{A} of the instrument shaft 1112 (shown in dashed)), whereas FIGS. 12B and 12C show the grasping portions 1115a, 1115b of the jaw members 1114a, 1114b in an open position and articulated in opposite directions of yaw or pitch relative to the longitudinal axis L_{A}. While various components of the jaw members 1114 and pulley and linkage wrist mechanism 1113 have a similar construction as that of the embodiment of FIGS. 8A-10D, the various clip guidance and retention features discussed could be applied to other embodiments of those components without departing from the scope of the present disclosure and claims.

As shown, the grasping portions 1115a, 1115b of the jaw members 1114a, 1114b have a configuration that enables reliable advancement and retention of a medical clip (not shown in the views of FIGS. 12A-12C), such as medical clip 3000 discussed above with reference to FIG. 3A and also shown in the views of FIGS. 10A and 10B, from proximal of the grasping portions 1115a, 1115b (i.e., between the opposed connection portions, only 1116a shown in FIG. 12A). As shown and described above with reference to grasping portion 315b in FIG. 3A, the grasping portions 1115a, 1115b comprise a split configuration that defines a longitudinally extending channel 1125a, 1125b (partially shown in the views of FIGS. 12A and 12B) configured to respectively receive opposing arms of a medical clip as it is advanced between the grasping portions 1115a, 1115b of the jaw members 1114a, 1114b. Inner lateral surfaces of the grasping portions 1115a, 1115b (the inner lateral surfaces facing each other so as to grasp material between the grasping portions 1115a, 1115b) are provided with radially protruding ledge features 1127a, 1127b that together with the upper and lower wall portions of the grasping portions 1115a, 1115b (referring to the respective upper and lower wall portions 1115Ua, 1115Lb of the grasping portions 1115a, 1115b that face away from each other) define guidance tracks 1124a, 1124b configured to receive respective pairs of retention bosses (such as retention bosses 3002a, 3002b of medical clip 3000), that are provided near or as part of the free, latching ends of the arms of the clip, for example, as described above with reference to FIG. 3.

In addition, the grasping portions 1115a, 1115b comprise leaf springs 1126a, 1126b (with opposing leaf springs on the opposite lateral side not shown in the cutaway view of FIGS. 12A-12c) on either side of the respective channels 1125a, 1125b. The leaf springs 1126a, 1126b (and corresponding opposing leaf springs) are biased inwardly (e.g., toward the centerline of each channel) to provide a force to retain the clip arms in the respective channels 1125a, 1125b during advancement of the clip. The inner surfaces of the leaf springs 1126a, 1126b (and the opposing set similar to 326a′, 326b′ not depicted in the cutaway views of FIGS. 12A-12C) further define small, recessed regions 1128a, 1128b (opposing recessed regions like 328a′, 328b′ not shown) that are configured to receive and, together with a curved distal end rigid portions of the grasping portions 1115a, 1115b provide stop surfaces for retaining the bosses of the clip and preventing longitudinal movement, particularly distally. Upon closure and latching of the clip by grasping portions 1115a, 1115b of the jaw members 1114a, 1114b, the latching force of the latching distal end portions of the clip (such as clip 3000 is sufficient to overcome the retention force of the grasping portions 1115a, 1115b acting on the clip, allowing it to be released therefrom.

As depicted in FIGS. 12A-12C, other optional guidance features may also be provided along various portions of the clip advancement path through the clevis and connection portions of the jaw members 1114a, 1114b (e.g., through the wrist mechanism 1113). For example, again with reference to the connection portion 1116a of jaw member 1114a shown in FIGS. 12A-12C, with the connection portion of the jaw member 1114b having a similar but mirror-image configuration across the longitudinal centerline of the wrist mechanism 1113 and jaw members 1114a, 1114b, the connection portion 1116a may also be provided with one or both of a central guidance ledge 1129a (1129b of connection portion of jaw member 1114b shown in FIG. 12C) and a peripheral guidance ledge 1130a that protrude slightly radially inwardly from the inner facing surfaces of the connection portion 1116a. The central guidance ledges 1129a, 1129b may extend longitudinally and distally from roughly a middle region of the connection portions 1116a (other connection portion not shown in FIGS. 12A-12C) and join with (be part of a length of) the ledge 1127a, 1127b or terminate close thereto. The peripheral guidance ledge 1130a is provided along a perimeter portion of the connection portion 1116a and extends to meet with upper surface 1115Ua of jaw member 1114a proximal to the locations of the slot 1117a that receives the pivot pin 1120a. A similar peripheral guidance ledge is also provided on the connection portion of the jaw 1114b (not shown). The guidance ledges of the connection portions of the jaw members 1114a, 1114b can help to define an overall guidance track system to receive the bosses of the clip arms, a part of such guidance track being the guidance tracks discussed above that extend along the length of the grasping portions 1115a, 1115b. Proximal portions of the guidance tracks 1124a, 1124b between the guidance ledges 1129a, 1129b, and 1130a may be wider and then taper distally to provide a large region for initial receipt and guidance of the bosses on the clips. The guidance ledge 1130a can have a ramped surface 1130Sa that presents as a “wall” in one yaw or pitch orientation of the wrist mechanism 1113, as depicted in FIG. 12B, and a “ramp” in the opposite yaw or pitch orientation of the wrist mechanism, as depicted in FIG. 12C.

In other embodiments, a flexible yet durable ribbon structure may be used as a guide feature along outer peripheral portions extending between the clevis and the jaw members. Referring to FIG. 13, an embodiment of an end effector having a pulley and linkage system wrist mechanism and jaw members configuration similar to that described with respect to the embodiment of FIG. 11 is depicted with such guidance features. As shown elongated, planar, flexible ribbon structures 1330a, 1330b, which may in various embodiments be metallic may be inserted in tracks provided 1334a, 1334b in the grasping portions 1315a, 1315b of the jaw members 1314a, 1314b. The ribbon structures 1330a, 1330b may extend proximally past the connection portions (connection portion 1316a of jaw member 1314a shown) and be coupled to the clevis 1319. In the embodiment of FIG. 13, the coupling to the clevis 1319 occurs via a tab portion 1335a, 1335b of the ribbon structure 1330a, 1330b being received in an aperture in a portion of the clevis 1319. As can be seen in FIG. 13, the length of the ribbon structure 1330a, 1330b captured in the tracks 1334a, 1334b is sufficient to maintain the positioning of the ribbon structure 1330a, 1330b through yaw or pitch articulation of the wrist mechanism 1313 and pivoting of the jaw members 1314a, 1314b to open/close the grasping portions 1315a, 1315b, thereby allowing the ribbon structures 1330a, 1330b to serve as guidance features as a clip is advanced through the end effector to be received by the grasping portion 1315a, 1315b of the jaw members 1314a, 1314b.

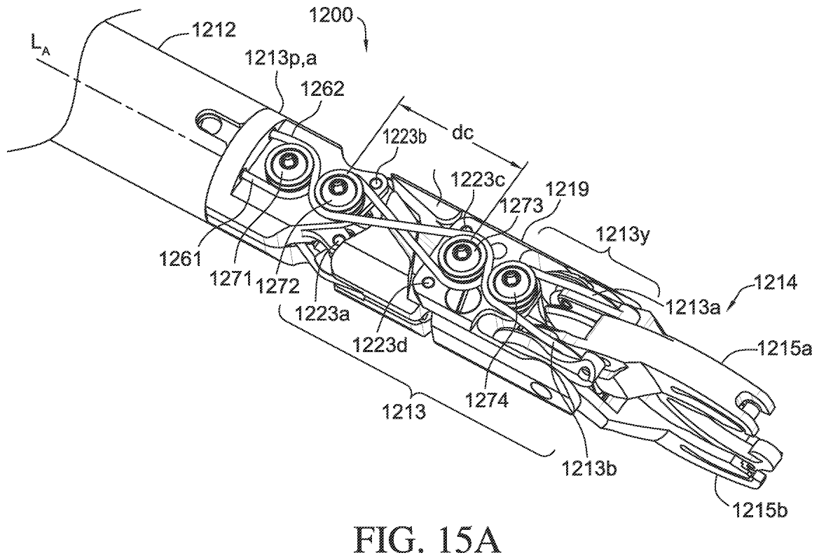

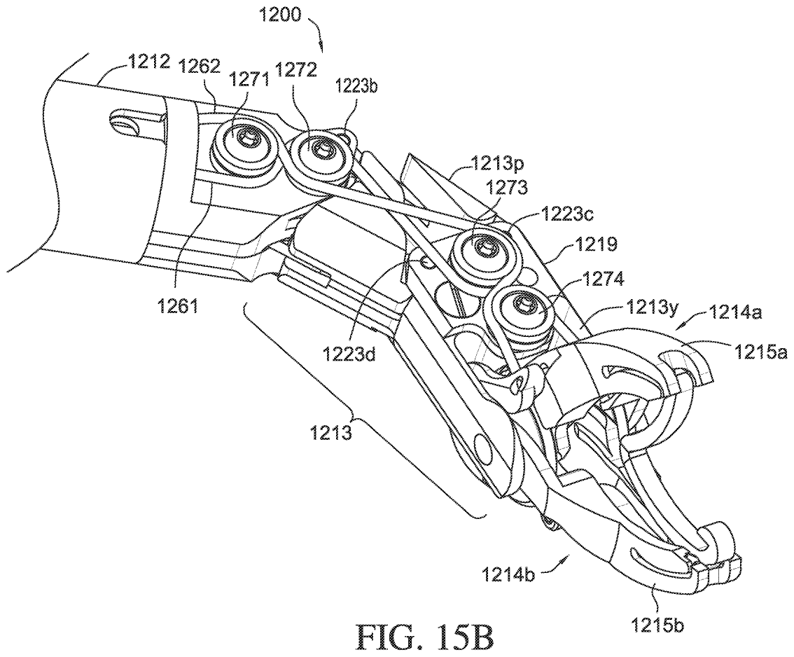

The ribbon structures 1330a, 1330b also provide a smooth surface extending over various components that may have gaps therebetween in particular orientations of the wrist mechanism 1313 and jaw members 1314a, 1314b., which can further assist in reducing a risk of a clip catching as it travels past such gaps between components. Similar ribbon structures could be used in the pitch wrist mechanisms such as in lieu of the tubular insert described above with reference to the embodiment of FIGS. 4A and 4B, or in the embodiments of the wrist structures of FIGS. 6 and 7 described above, and FIGS. 15-17 described below. Reference is made to FIG. 14 depicting an embodiment utilizing ribbon structures 1430a, 1430b similar to ribbon structures 1330a, 1330b but extending through a wrist mechanism (e.g., pitch articulatable wrist mechanism) that attaches between an instrument shaft 1412 and an end effector 1414 (partially shown). The wrist mechanism 1413 may be similar to the pitch wrist mechanisms described below with reference to the embodiments of FIGS. 15-18, though they are not limited to use with such wrist mechanism configurations and can be employed in other wrist mechanism configurations.

In various embodiments, the clevis of the end effector also may be provided with guidance features to assist with maintaining a desired orientation of the clip as it advances through the clevis to the wrist mechanism. The clevis guidance features in some embodiments may be configured to interact with those of the connection portions. Again referring to the sectional views of FIGS. 12A-12C, with only a portion of the clevis 1119 depicted for simplicity (the other portion being a mirror image along the longitudinal centerline L_{A}, the clevis 1119 has a generally open (hollow) cross-section that allows for the clip to be received and advanced therethrough, with the interior distal end portion of the clevis 1119 from which axial location the clevis extensions extend, having a concave configuration that abuts and provides a complementary mating interface with the rounded profile of the perimeter of the connection portions (shown with respect to connection portion 1116a in the figures) of the jaw members 1114a, 1114b. The complementary and abutting configuration, as well as the overall rounded perimeter of the connection portions of the jaw members 1114a, 1114b allows for minimizing gaps between the wrist component and the clevis, regardless of the yaw or pitch orientation of the wrist mechanisms or the open/close pivot configuration of the jaw members, thereby assisting in a reliable advancement of the clip through the clevis 1119 to the wrist mechanism 1113 so as to reduce the potential risk of the clip being caught on a surface or between components.

The inner surface of the clevis 1119 is also provided with a guidance ledge lip 1132 around the circumference and an axially extending and radially protruding longitudinally extending ledge 1131 disposed so that an upper surface 1131U of the ledge 1131 is generally along a centerline that approximately intersects with the rotational axis R_{A} of the pulleys (not shown in FIGS. 12A-12C). The ledge 1131, along with the inner wall and lip 1132 of the clevis 1119 defines two chutes 1133a, 1133b (with similar but opposing chutes being defined by the opposite side of the clevis 1119, which can respectively help to guide the bosses on the arms of a medical clip, such as medical clip 3000 discussed above with respect to various embodiments. A distal end portion of the ledge 1131 terminates along the connections portion 1116a (and similar on connection portion of jaw member 1114b not shown), for example at a location slightly proximal to central guidance ledges 1129a 1129b (shown in FIG. 12C). This interaction and configuration can help to provide a guidance track for the bosses on the upper and lower arms of the clip, such as clip 3000, from the clevis 1119 through the wrist mechanism 1113, helping to maintain the general desired orientation of the clip arms into the respective grasping portions 1115a, 1115b of the jaw members 1114a, 1114b. In embodiments where the connection portions and the clevis have guidance ledges, e.g., as depicted in FIGS. 12A-12C, the various guidance ledges (e.g., 1129a, 1129b, 1130a, and 1131, long with the interior wall surfaces and lip 1132 of the clevis 1119 can be configured and arranged to interact with each other in the various yaw or pitch orientations of the wrist and pivot orientations of the jaw members, as reflected in the oppositely articulated states shown in FIGS. 12B and 12C, so as to provide desired guidance and reliable advancement of the clip regardless of these orientations.

While FIGS. 12A-12C illustrate an embodiment in which each of the clevis, wrist mechanism (via the connections portions of the jaw members), and grasping portions of the jaw members are provided with the guidance features, the present disclosure contemplates instruments that include one or more of any of these guidance features. Moreover, in view of the teachings herein, those of ordinary skill in the art would understand how to utilize other configurations of guidance features, such as the ribbon structures discussed above or other type structures, serving similar functions and selected based on the particular clip configuration for which the instruments may be used, without departing from the scope of the present disclosure.

Various additional embodiments of the present disclosure contemplate instruments that are configured to apply medical clips from proximal an end effector into the jaw members and that comprises a wrist mechanism configured to articulate in both pitch and yaw (two degrees of freedom of articulation) relative to a longitudinal axis of shaft of the instrument. In various embodiments, the wrist mechanism providing for articulation in both pitch and yaw is further configured to define a medical clip advancement path therethrough, regardless of the articulation state; such embodiments can therefore provide for multiple clips to be stored proximal the wrist mechanism and advanced consecutively through the wrist mechanism to the jaw members for application intraoperatively.

One embodiment of such an instrument is depicted in the partial perspective views of FIGS. 15A-15B, which show the distal end portion of an instrument shaft, wrist mechanism, and end effector, with FIG. 15A depicting the wrist mechanism in a neutral configuration relative to the longitudinal axis of the instrument shaft and the grasping portions of the jaw members in a closed position, and FIG. 15B depicting the wrist mechanism articulated in pitch and yaw and the grasping portions of the jaw members in a partially open position.

With reference to FIGS. 15A and 15B, the instrument 1200 comprises a wrist mechanism 1213 coupling the end effector 1214 and clevis 1219 to the instrument shaft 1212. The wrist mechanism 1213 combines a crossed four-bar linkage system type wrist mechanism 1213p to provide the pitch articulation (having a similar operation and structure as described above with reference to FIGS. 5 and 6) and a pulley and linkage system type wrist mechanism 1213y to provide the yaw articulation and having a configuration as described above with reference to wrist mechanisms in the embodiment of FIGS. 8-11 described above. The pulley and linkage system of the wrist mechanism 1213y also provides the pivoting of the jaw members 1214a, 1214b relative to each other to provide the open/close degree of freedom in a manner as described above with reference to FIGS. 8-11. For the purposes of simplification of the description, the pulley and linkage wrist mechanism 1213y are not described again here.

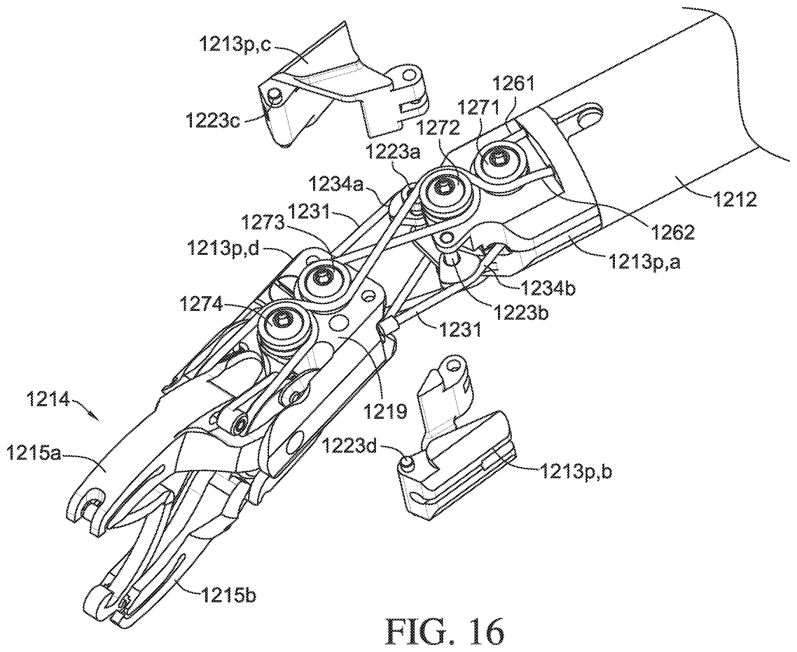

While the crossed four-bar linkage pitch wrist mechanism 1213p operates under similar principles to that described above with respect to the embodiments of FIGS. 5 and 6, reference is made to FIG. 16, which shows an exploded view of the wrist mechanism 1213p to better understand the construction and operation of the wrist mechanism 1213p. As shown in FIG. 16, the crossed four-bar linkage that makes up the pitch wrist mechanism 1213p includes two cross links 1213p,b and 1213p,c (similar to links 413b, 413c of the embodiment of FIGS. 5 and 6) pivotably coupled via pivot pins 1223a, 1223b at one end to an adaptor link 1213p,a, which is fixedly attached to the instrument shaft 1212 (similar to link 413a of the embodiment of FIGS. 5 and 6) and at their opposite ends via pivot pins 1223c, 1223d to the proximal end portion of the clevis 1219, which serves as the fourth link 1213p,d (similar to link 413d of the embodiment of FIGS. 5 and 6) of the crossed four-bar linkage.

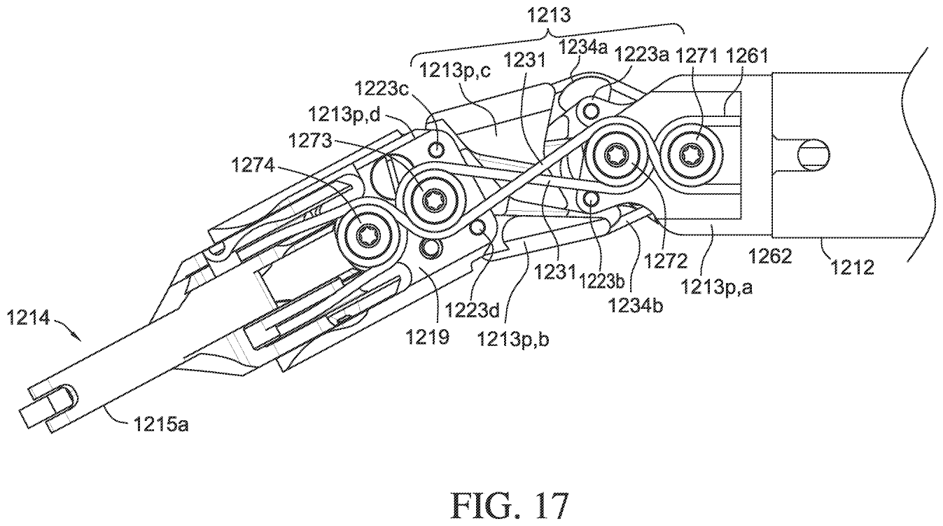

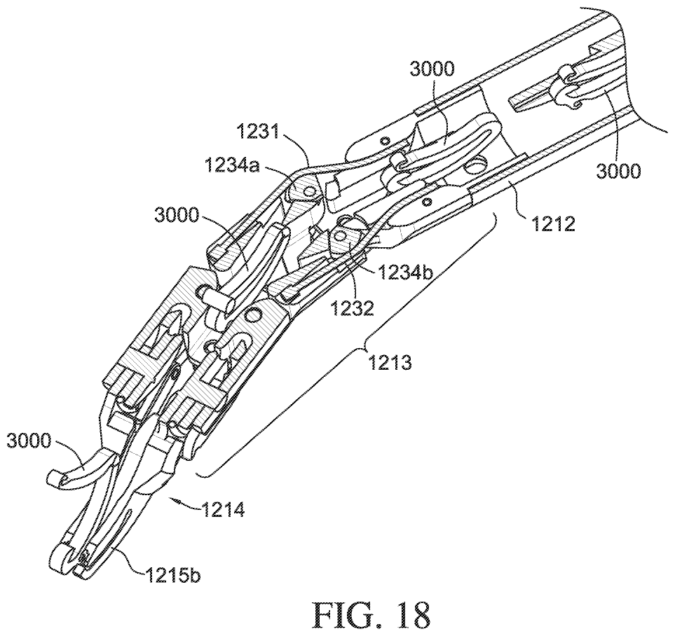

As shown in FIGS. 17 and 18, the form of which is a top plan view of FIG. 16 (with respect to the orientation of that figure) but with the crossed four-bar linkage shown transparent and not exploded, and the latter of which is a partial, longitudinal sectional view, both to provide a better view of the actuation members 1231, 1232 operably coupled to actuate the pitch articulation of the crossed four-bar linkage wrist mechanism 1213p. As shown, oppositely disposed actuation members 1231, 1232 are depicted and extend through the shaft 1212 from the proximal end force transmission mechanism (not shown) through the adapter link 1213a, around partial guide pulleys 1234a, 1234b that are coupled to the pivot pins 1223a, 1223b and then to the proximal end portion of clevis 1219, where the actuation members 1231, 1232 are fixedly coupled, for example via a swage coupling or the like. As described above, the actuation members 1231, 1232 may be cables or of similar construction so as to be able to be paid in (pulled) to create a tension force that pulls on the side of the link 1213p, d to which the relevant actuation member 12311, 1232 is attached. By paying out the cable 1231, 1232 opposite to the one being placed in tension the crossed four-bar linkage wrist mechanism 1213 can articulate the end effector 1214 relative to the instrument shaft 1212 toward the side of the actuation member 1231, 1232 being pulled. The guide pulleys 1234a, 1234b route the actuation members 1231, 1232 toward opposite outer peripheral sides of the wrist mechanism 1213p such that as they extend through the pitch wrist mechanism 1213p they are routed internally but along outer respective sides of the links 1213b, 1213c. Similarly, the actuation members 1231, 1232 couple to the clevis 1219 (link 1213p, d) toward an outer periphery. This routing allows the actuation members 1231, 1232 to remain clear of the central interior portion of the wrist mechanism 1213p to allow for advancement of a medical clip therethrough.

To further allow sufficient room to advance a clip through the wrist mechanism 1213 and into position between the grasping portions 1215a, 1215b of the jaw members 1214a, 1214b, actuation members of the instrument 1200 that control the pulley and linkage system of yaw wrist mechanism 1213y are routed externally of the wrist mechanism 1213, clevis 1219, and the adapter link 1213p, a of the pitch wrist mechanism 1213p. FIGS. 15-18 show various views of the pair of actuation members 1261, 1262, which can be cables or similar construction, operably coupled to and extending from the force transmission mechanism (not shown) down the instrument shaft 1212 where they exit the instrument shaft 1212 at the adapter link 1213p, a. A pair of proximal pulleys 1271, 1272 are disposed adjacent to each other and provided on an exterior of the link 1213p, a. The pair of proximal pulleys 1271, 1272 are positioned in a proximal-distal relationship along the longitudinal direction and have rotational axes that are parallel to each other and perpendicular to the longitudinal axis of the shaft L_{A}.

A mirrored set of pulleys is on the opposite side of the link 1213p, a (not shown in the view of FIGS. 15-18). The pulleys 1271, 1272 are also positioned such that their rotational axes are generally along a longitudinal centerline of the exterior of the shaft 1212, (e.g., so that the rotational axes intersect the longitudinal axis L_{A} of the shaft 1212). Each actuation member 1261, 1262 extends from the shaft 1212 partially around pulley 1271 on opposite sides, crosses over the other, and then partially wraps around the pulley 1272 on opposite sides again. From the pair of proximal pulleys 1271, 1272, the actuation members 1261, 1262 again cross each other at a location over the cross bar links 1213p, b, 1213p, c of the pitch wrist mechanism 1213p and extend to partially wrap around a pair of distal pulleys 1273, 1274. The pair of distal pulleys 1273, 1274 are disposed adjacent to each other and provided on an exterior of the clevis 1219 to which the components of wrist mechanism 1213y and jaw members 1214a, 1214b are attached. The pair of distal pulleys 1273, 1274, like the pair of proximal pulleys 1271, 1272, are positioned in a proximal-distal relationship along the longitudinal direction of the clevis 1219 and have rotational axes that are parallel to each other and perpendicular to the longitudinal axis of the shaft L_{A} in the neutral articulation state of the wrist mechanism 1213 depicted in FIG. 15A. The pair of distal pulleys 1273, 1274 are also positioned such that their rotational axes are generally along a longitudinal centerline of the exterior of the shaft in the neutral articulation state of the wrist mechanism 1213, (e.g., so that the rotational axes intersect the longitudinal axis L_{A} of the shaft 1212). A mirrored pair of pulleys is on the opposite side of the clevis (not shown in the figures).

Similar to the routing of the actuation members 1261, 1262 over the proximal pair of pulleys 1271, 1272, each actuation member 1261, 1262 extends from where they cross over the links 1213p, b, 1213p, c partially around pulley 1273 on opposite sides, cross over each other again, and then partially wrap around the pulley 1274 on opposite sides again. From pulley 1274, the actuation members 1261, 1262 extend distally and generally parallel to each other to wrap around the pulleys 1213a, 1213b (as perhaps best shown in FIG. 16-18) of the pulley and linkage system of yaw wrist mechanism 1213y. The actuation members 1261, 1262 then wrap around in a mirrored way over pairs of proximal and distal pulleys disposed on an opposite side of the wrist mechanism 1213 (now shown in the views of FIGS. 15-18 as noted above) and are again routed through the shaft 1212 to operably couple with drive components of a force transmission mechanism (which can be manually operable and/or teleoperable via a manipulator system as mentioned).

The actuation member and pulley arrangement maintain a center to center distance d_{c}, regardless of the state of articulation of the pitch wrist mechanism 1213p, between the distal pulley 1272 of the pair of proximal pulleys and the proximal pulley 1273 of the pair of distal pulleys (and likewise for the set of actuation pulleys on the other side of the instrument not visible in FIGS. 15-17). By maintaining the distance d_{c}, the articulation of the pitch wrist mechanism 1213p in the pitch degree of freedom, and the other arrangement of the pulleys 1271, 1272, 1273, 1274 and routing pattern of the actuation members 1261, 1262 wrapping around the pulleys, length conservation of the actuation members can be realized so that the range of pitch articulation can be relatively independent of yaw articulation and jaw member pivoting that opens/closes the grasping portions 1215a, 1215b of jaw members 1214a, 1214b. In other words, the pitch articulation does not significantly, if at all, impact or cause actuation of the wrist mechanism 1213y to articulate in yaw or pivoting motion of the jaw members 1214a, 1214b.

FIG. 18 depicts how a medical clip 3000 (shown in 4 different locations of advancement, can be advanced through the distal end portion of the instrument shaft 1212 through the wrist mechanism 1213, and into position between the grasping portions 1215a, 1215b of the embodiment of FIGS. 15-18 (only 1215b being show in FIG. 18) of the end effector 1214.

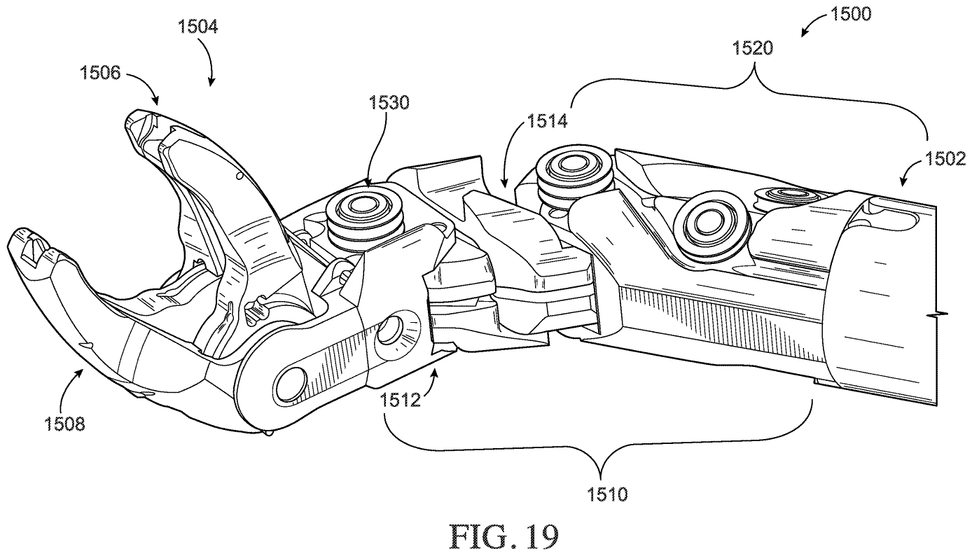

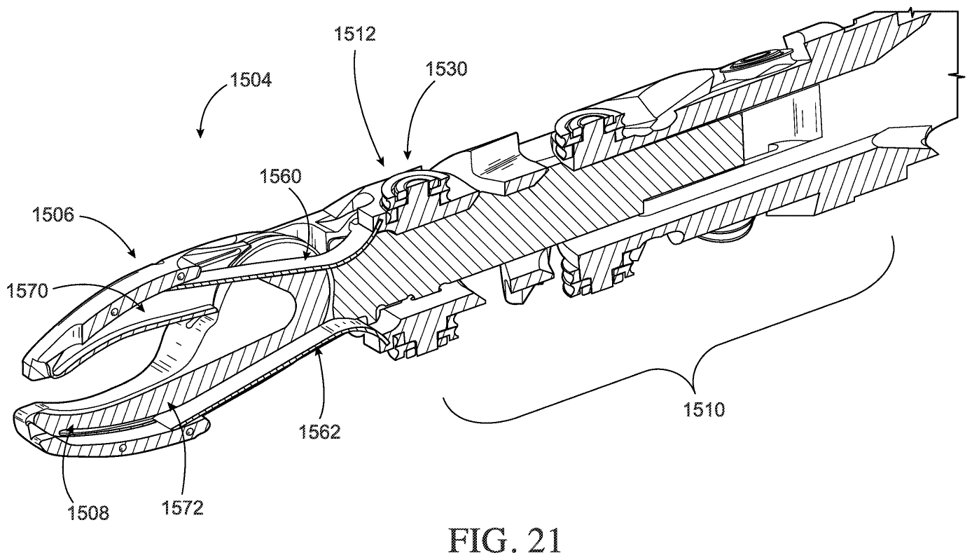

Referring now to FIGS. 19-21, another embodiment of an instrument for applying clips comprises a wrist mechanism 1510 coupling a clevis 1512 and end effector 1504 to the instrument shaft 1502 at a pivot point, such as a hinge 1514. The wrist mechanism 1510 comprises a pulley and linkage system type wrist mechanism 1520 to provide the yaw articulation and having a configuration as described above with reference to the wrist mechanisms in the embodiment of FIGS. 15-17 described above. Again, for definitional purposes, the “yaw” axis is defined as the axis that the end effector rotates around to move side to side relative to the upper and lower portions of the shaft and the “pitch” axis is defined as the axis that the end effector rotates around to move up and down relative to the shaft (similar to the pitch and yaw axis of an airplane). However, it will be understood that these definitions can be reversed. For the purposes of simplification of the description, the pulley and linkage wrist mechanism 1520 for yaw rotation is not described again here.

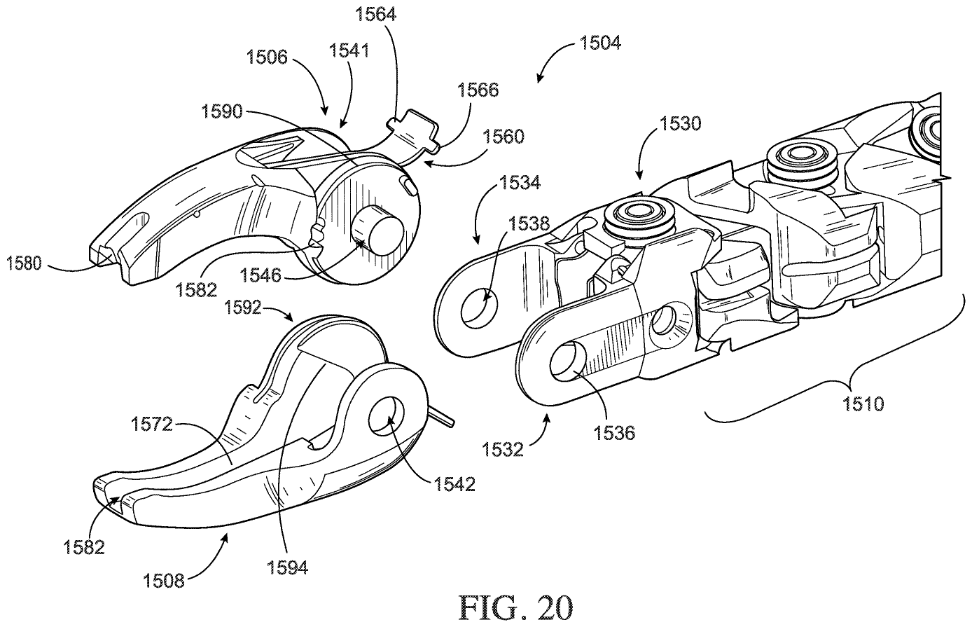

Similar to previous embodiments, end effector 1504 comprises first and second jaws 1506, 1508 that comprise a pair of opposing grasping portions that are configured to open and close via relative pivoting motion to receive and hold a clip and apply force during closure sufficient to latch the clip around an object. As shown in FIG. 20, clevis 1512 of end effector 1504 comprises first and second extension arms 1532, 1534 each having an opening 1536, 1538 for receiving respective axles 1546 extending laterally outward from jaws 1506, 1508. Note that only axle 1546 of jaw 1506 is shown in FIG. 20, although it will be understood that jaw 1508 contains a similar axle (not shown) extending through opening 1538 of extension arm 1534. Axle 1546 of jaw 1506 also extends through an opening 1542 in jaw 1508 (and the axle of jaw 1508 extends through a similar opening in jaw 1506) to rotatably couple jaws 1506, 1508 together. Jaws 1506, 1508 are pivotally coupled to extension arms 1534, 1536 by rotation of axles 1546 within openings 1536, 1538 such that the jaws may rotate about a pitch axis relative to the extension arms and wrist mechanism 1510.

End effector 1504 further includes a pulley 1530 having a link, such as a cable or band, (not shown) that extends around pulley 1530 and pulley drive surfaces 1590, 1592 of jaws 1506, 1508. Pulley 1530 and the cable drive function to actuate jaws 1506, 1508 to open and close the jaws, and may be suitably coupled to a proximal actuator as described above. Each of the jaws 1506, 1508 may further include a cable crimp pocket 1582 on drive surfaces 1590, 1592.

End effector 1504 further comprises first and second ribbons 1560, 1562 that function similar to 1330, 1330b described above. As shown, ribbons 1560, 1562, which may in various embodiments be metallic may be inserted in guide tracks 1570, 1572 provided in the grasping portions of the jaw members 1506, 1508. The ribbon structures 1560, 1562 may extend proximally past the connection portions and be coupled to the clevis 1512. The coupling to the clevis 1512 occurs via tab portions 1564, 1566 of the ribbon structure 1560, 1562 being received in an aperture (not shown) in a portion of the clevis 1319.

The length of the ribbons 1560, 1562 captured in the tracks 1570, 1572 is sufficient to maintain the positioning of the ribbons 1560, 1562 through yaw or pitch articulation of the wrist mechanism 1510 and pivoting of the jaw members 1506, 1508 to open/close, thereby allowing the ribbons 1560, 1562 to serve as guidance features as a clip is advanced through the end effector to be received by the grasping portion of the jaw members 1506, 1058. The ribbons 1560, 1562 also provide a smooth surface extending over various components that may have gaps therebetween in particular orientations of the wrist mechanism 1510 and jaw members 1506, 1508 which can further assist in reducing a risk of a clip catching as it travels past such gaps between components.

Guide tracks 1570, 1572 generally extend from a proximal portion of the jaws 1506, 1508 to the distal end of each jaw inside of ribbons 1560, 1562. Guide tracks 1570, 1572 and ribbons 1560, 1562 ensure that the arms of clip advancer (not shown) pass along guide tracks 1570, 1572 to distal ends of jaw members 1506, 1508 as the drive member is advanced distally into jaws 1506, 1508. A more complete description of a suitable clip advancer can be found in commonly assigned, co-pending U.S. Provisional Applications Ser. Nos. 63/505,738, 63/505,740, 63/505,742, 63/505,870, 63/505,875 and 63/505,735, filed Jun. 2, 2023, the complete disclosures of which have been previously incorporated herein. The tracks 1570, 1572 may further comprise a clip track divider 1594 that separates the individual guide tracks 1570, 1572.

Similar to previous embodiments, the grasping portions of each jaw 1506, 1508 comprise a split configuration that defines a longitudinally extending channel 1580, 1582 (see FIG. 20) configured to respectively receive opposing arms of a medical clip as it is advanced between the grasping portions of the jaw members 1506, 1508.

Various embodiments of instruments described herein may be configured to apply medical clips commercially available from Teleflex®, such as Weck® Hem-O-Lok polymer clips. However, such clips are not limiting and those of ordinary skill in the art would appreciate that the instruments disclosed herein could be utilized to apply clips having other configurations (including the clips described in the above-referenced commonly assigned patent applications), with the principles of operation and design disclosed herein providing guidance for any modifications that may be needed to effect the same.

The embodiments described herein may be well suited for use in medical applications. In particular, some embodiments are suitable for use in, for example, surgical, teleoperated surgical, diagnostic, therapeutic, and/or biopsy procedures. Such procedures could be performed, for example, on human patients, animal patients, human cadavers, animal cadavers, and portions or human or animal anatomy. Some embodiments may also be suitable for use in, for example, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, including applying clips to, non-tissue work pieces at remote worksites, such as tubes and the like that may need to be closed with a clip. In non-limiting embodiments, the techniques, methods, and devices described herein may be used in, or may be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the da Vinci® Surgical Systems are illustrative and not to be considered as limiting the scope of the disclosure herein.

Of course, it will be recognized that that the drive member and clips may be advanced (and the drive member retracted) through the wrist to the end effector while the wrist is bent (i.e., while the end effector is rotated in the yaw, pitch or roll directions). As discussed above, the drive members described herein include flexible portions that bend or flex within the wrist of the instrument to allow the drive member to remain positioned in the wrist and the jaws during articulation of the end effector. Thus, in certain embodiments, the control system may be operated to first articulate the end effector such that the jaws are positioned around the target tissue or vessel and then advance the drive member and the first clip into the jaws.

As used herein and in the claims, terms such as computer-assisted or teleoperable in referencing manipulator systems, or the like should be understood to refer broadly to any system comprising one or more controllable kinematic structures (“manipulators”) that are movable and controllable at least in part through the aid of an electronic controller (with or without human inputs). Such systems may occasionally be referred to in the art and in common usage as robotically assisted systems or robotic systems. Such systems include systems that are controlled by a user (for example through teleoperation), by a computer automatically (so-called autonomous control), or by some combination of these. In examples in which a user controls at least some of the operations of the manipulator, an electronic controller (e.g., a computer) may facilitate or assist in the operation. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems may be implemented in a variety of contexts to perform a variety of procedures, both medical and non-medical. Thus, although some examples described in greater detail herein may be focused on a medical context, the devices and principles described herein are also applicable to other contexts, such as industrial manipulator systems.

This description and the accompanying drawings that illustrate various aspects and embodiments should not be taken as limiting—the claims define the scope of protection. Various mechanical, compositional, structural, electrical, and operational changes may be made without departing from the spirit and scope of this description and the claims. In some instances, well-known structures, components, and techniques have not been shown or described in detail in order not to obscure the present disclosure. Like numbers in two or more figures that end in the same two digits but begin with a different series, such as 8xx, 10xx, etc. are as best as possible used to represent the same or similar elements.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment

Further, this description's terminology is not intended to be limiting. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes includes various special device positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present description is intended to embrace all such alternatives, modifications, and variances. As well, one skilled in the art will appreciate further features and advantages of the present disclosure based on the above-described embodiments. Accordingly, the present description is not to be limited by what has been particularly shown and described, except as indicated by the appended claims.

For example, in a first aspect, a first embodiment is surgical instrument for applying surgical clips to tissue. The instrument comprises an elongate shaft having a longitudinal axis; an end effector on a distal end of the shaft including first and second jaws; an articulation assembly coupled to the first and second jaws and configured to move the first and second jaws between an open position and a closed position; and a drive member configured to translate longitudinal through the articulation assembly to deliver one or more surgical clips to the first and second jaws.

A second embodiment is the first embodiment, wherein the articulation assembly comprises a pulley rotatable about an axis substantially perpendicular to the longitudinal axis, and a band around the pulley and coupled to the first and second jaws.

A third embodiment is any combination of the first two embodiments, wherein rotation of the pulley in a first direction causes the first and second jaws to move away from each other.

A 4^{th} embodiment is any combination of the first 3 embodiments, wherein rotation of the pulley in a second direction causes the first and second jaws to move towards each other.

A 5^{th} embodiment is any combination of the first 4 embodiments, wherein the first and second jaws each comprise a curved drive surface and wherein the band extends along the curved drive surfaces.

A 6^{th} embodiment is any combination of the first 5 embodiments, wherein the articulation assembly comprises first and second extension members each having an opening, and each of the first and second jaws comprises an axle disposed within one of the openings.

A 7^{th} embodiment is any combination of the first 6 embodiments, wherein each of the jaws comprises a lateral surface with an opening, wherein the axle of the first jaw extends through the opening in the second jaw and the axle of the second jaw extends through the opening in the first jaw.

An 8^{th} embodiment is any combination of the first 7 embodiments, further comprising a wrist assembly comprising an internal channel for advancing the drive member therethrough, and first and second pulleys disposed laterally outward from the internal channel and rotatable about an axis substantially perpendicular to the longitudinal axis.

A 9^{th} embodiment is any combination of the first 8 embodiments, wherein the wrist assembly further comprising a first band around at least a portion of the first pulley and a second band around at least a portion of the second pulley, the bands operable to rotate the pulleys upon longitudinal translation of a proximal end portion of the bands.

A 10^{th} embodiment is any combination of the first 9 embodiments, wherein the articulation assembly is disposed laterally outward from the first and second jaws relative to the longitudinal axis of the shaft.

A 11^{th} embodiment is any combination of the first 10 embodiments, wherein rotation of the first pulley in a first direction causes the first jaw to move away from the second jaw and rotation of the second pully in a second direction opposite to the first direction causes the second jaw to move away from the first jaw.

A 12^{th} embodiment is any combination of the first 11 embodiments, wherein the axis is a ya2 axis relative to the shaft.

A 13^{th} embodiment is any combination of the first 12 embodiments, further comprising a first link having a proximal end coupled to the first pulley and a distal end coupled to the first jaw, and a second link having a proximal end coupled to the second pulley and a distal end coupled to the first jaw.

An 14^{th} embodiment is any combination of the first 13 embodiments, wherein the first and second links are disposed laterally outward from the first and second jaws.

A 15^{th} embodiment is any combination of the first 14 embodiments, wherein the second link has a longer length than the first link.

A 16^{th} embodiment is any combination of the first 15 embodiments, wherein the first link is substantially linear and the second link comprises a distal linear portion and a proximal curved portion.

A 17^{th} embodiment is any combination of the first 16 embodiments, further comprising a pivot pin extending through the first jaw, wherein the pivot pin is coupled to the first and second links.

A 18^{th} embodiment is any combination of the first 17 embodiments, further comprising a third link having a proximal end coupled to the second pulley and a distal end coupled to the second jaw, and a fourth link having a proximal end coupled to the first pulley and a distal end coupled to the second jaw.

A 19^{th} embodiment is any combination of the first 18 embodiments, wherein the third and fourth links are disposed laterally outward from the first and second jaws.

A 20^{th} embodiment is any combination of the first 19 embodiments, wherein the fourth link has a longer length than the third link.

An 21^{st} embodiment is any combination of the first 20 embodiments, wherein the third link is substantially linear and the fourth link comprises a distal linear portion and a proximal curved portion.

A 22^{nd} embodiment is any combination of the first 21 embodiments, further comprising an actuator extending through the articulation assembly lateral of the drive member and being configured to move the jaws between the open and closed positions.

A 23^{rd} embodiment is any combination of the first 22 embodiments, wherein the drive member comprises a distal component for removably coupling to a surgical clip.

A 24^{th} embodiment is any combination of the first 23 embodiments, wherein the first and second jaws include first and second guide tracks, respectively, for receiving protrusions on the surgical clip to guide the surgical clip to the jaws.

A 25^{th} embodiment is any combination of the first 24 embodiments, wherein the surgical clip includes first and second arms, and wherein at least one of the first and second jaws comprises an engagement element for securing at least one of the first and second arms of the clip to said at least one of the first and second jaws.

In another aspect, a first embodiment is a surgical instrument for applying surgical clips to tissue. The instrument comprises an elongate shaft having a longitudinal axis; an end effector on a distal end of the shaft including first and second jaws; an articulation assembly coupled to the first and second jaws and configured to move the first and second jaws between an open position and a closed position, the articulation assembly comprising an internal longitudinal channel; and a drive member configured to translate longitudinal through the internal channel of the articulation assembly to deliver one or more surgical clips to the first and second jaws.

A second embodiment is the first embodiment, wherein the articulation assembly comprises first and second cam pins coupled to the first and second jaws, respectively.

A third embodiment is any combination of the first two embodiments, further comprising first and second connection portions coupled to either side of the first jaw, the first and second connection portions each comprising a cam slot for receiving the first cam pin.

A 4^{th} embodiment is any combination of the first 3 embodiments, further comprising third and fourth connection portions coupled to either side of the second jaw, the third and fourth connection portions each comprising a cam slot for receiving the second cam pin.

A 5^{th} embodiment is any combination of the first 4 embodiments, wherein the first, second, third and fourth connection portions are disposed laterally outward from the first and second jaws relative to the longitudinal axis.

A 6^{th} embodiment is any combination of the first 5 embodiments, further comprising first and second plates coupled to the shaft and disposed laterally outward from the first and second jaws, where the first and third connection portions are pivotally coupled to the first plate and the second and fourth connection portions are pivotally coupled to the second plate.

A 7^{th} embodiment is any combination of the first 6 embodiments, wherein the cam slots are non-linear.

An 8^{th} embodiment is any combination of the first 7 embodiments, further comprising an actuator extending through the articulation assembly lateral of the drive member and being configured to move the jaws between the open and closed positions.

A 9^{th} embodiment is any combination of the first 8 embodiments, wherein the drive member comprises a distal component for removably coupling to a surgical clip.

A 10^{th} embodiment is any combination of the first 9 embodiments, wherein the first and second jaws include first and second guide tracks, respectively, for receiving protrusions on the surgical clip to guide the surgical clip to the jaws.

An 11^{th} embodiment is any combination of the first 10 embodiments, wherein the surgical clip includes first and second arms, and wherein at least one of the first and second jaws comprises an engagement element for securing at least one of the first and second arms of the clip to said at least one of the first and second jaws.

A 12^{th} embodiment is any combination of the first 11 embodiments, further comprising a wrist assembly coupling the end effector to the shaft, the wrist assembly comprising an internal channel for receiving the drive member and first and second links coupled to each other by a joint.

A 13^{th} embodiment is any combination of the first 12 embodiments, wherein the wrist assembly comprises first and second links pivotally coupled to each other about a joint, wherein the first and second links and the joint are disposed laterally outward of the internal channel of the wrist assembly.

A 14^{th} embodiment is any combination of the first 13 embodiments, wherein the first link is coupled to the end effector and the second link is disposed proximal of the first link, the wrist assembly further comprising a third link pivotally coupled to the second link about a second joint, wherein the third link and the second joint are disposed laterally outward of the internal channel of the wrist assembly.

In another aspect, a first embodiment is a surgical instrument for applying surgical clips to tissue. The instrument comprises an elongate shaft having a longitudinal axis; an end effector on a distal end of the shaft including first and second jaws; a wrist assembly coupling the end effector to the shaft for rotating the end effector about an axis substantially perpendicular to the longitudinal axis; and a drive member configured to translate longitudinal through the wrist assembly to deliver one or more surgical clips to the first and second jaws.

A second embodiment is the first embodiment, wherein the wrist assembly comprises an internal channel and a first link extending circumferentially around the internal channel from a first end to a second end, wherein the first end is proximal to the second end.

A 3^{rd} embodiment is any combination of the first 2 embodiments, wherein the wrist assembly a second link extending circumferentially around the internal channel from a first end to a second end, wherein the first end is proximal to the second end.

A 4^{th} embodiment is any combination of the first 3 embodiments, wherein the first end of the first link is substantially circumferentially aligned with the second end of the second link and the second end of the first link is substantially circumferentially aligned with the first end of the second link.

A 5^{th} embodiment is any combination of the first 4 embodiments, wherein the first and second links are movable to allow rotation of the end effector relative to the shaft.

A 6^{th} embodiment is any combination of the first 5 embodiments, further comprising an articulation assembly coupled to the first and second jaws and configured to move the first and second jaws between an open position and a closed position, wherein the articulation assembly comprises an internal channel and the drive member is configured to translate longitudinally through the internal channel.

A 7^{th} embodiment is any combination of the first 6 embodiments, further comprising an actuator extending through the wrist assembly lateral of the drive member and being configured to move the jaws between the open and closed positions.

An 8^{th} embodiment is any combination of the first 7 embodiments, wherein the drive member comprises a distal component for removably coupling to a surgical clip.

A 9^{th} embodiment is any combination of the first 8 embodiments, wherein the wrist assembly comprises an internal channel first and second pulleys disposed laterally outward from the internal channel and rotatable about an axis substantially perpendicular to the longitudinal axis.

A 10^{th} embodiment is any combination of the first 9 embodiments, further comprising a first band around at least a portion of the first pulley and a second band around at least a portion of the second pulley, the bands operable to rotate the pulleys upon longitudinal translation of a proximal end portion of the bands.

1. A surgical instrument for applying surgical clips to tissue, the instrument comprising:
an elongate shaft having a longitudinal axis;
an end effector on a distal end of the shaft including a first jaw and a second jaw;
an articulation assembly coupling the end effector to the shaft, the articulation assembly comprising a pulley rotatable about an axis substantially perpendicular to the longitudinal axis and a cable extending around at least a portion of the pulley, the cable coupled to the first and second jaws and configured to move at least one of the first and second jaws between an open position and a closed position; and
a drive member configured to translate longitudinal through the articulation assembly to deliver a surgical clip to the first and second jaws.

2. (canceled)

3. The surgical instrument of claim 1, wherein translation of the cable in a first direction causes the first and second jaws to move away from each other.

4. The surgical instrument of claim 3, wherein translation of the cable in a second direction causes the first and second jaws to move towards each other.

5. The surgical instrument of claim 1, wherein the first and second jaws each comprise a curved drive surface and wherein the cable extends along the curved drive surfaces.

6. The surgical instrument of claim 1, wherein the articulation assembly comprises first and second extension members each having an opening, and each of the first and second jaws comprises an axle disposed within one of the openings.

7. The surgical instrument of claim 6, wherein each of the jaws comprises a lateral surface with an opening, wherein the axle of the first jaw extends through the opening in the second jaw and the axle of the second jaw extends through the opening in the first jaw.

8. The surgical instrument of claim 1, wherein the articulation assembly comprises an internal channel for advancing the drive member therethrough, wherein the pulley is a first pulley, the instrument further comprising a second pulley disposed laterally outward from the internal channel and rotatable about an axis substantially perpendicular to the longitudinal axis.

9. The surgical instrument of claim 8, wherein the cable comprises a first cable, and the articulation assembly further comprises a second cable extending around at least a portion of the second pulley.

10. The surgical instrument of claim 9, further comprising a third pulley and a fourth pulley, wherein the first pulley is disposed on the end effector and the second, third and fourth pulleys are disposed on the shaft.

11. The surgical instrument of claim 9, wherein the first and second cables are configured to rotate the first and second jaws about a yaw axis substantially perpendicular to the longitudinal axis.

12. (canceled)

13. The surgical instrument of claim 11, wherein the articulation assembly comprises a first link and a first actuation member coupled to the first link and a second link and a second actuation member coupled to the second link, wherein the first and second actuation members are configured to rotate the first and second jaws about a pitch axis substantially perpendicular to the yaw axis and the longitudinal axis.

14. The surgical instrument of claim 13, wherein the first and second links comprise first and second cross links extending from a first side of the longitudinal axis to a second side of the longitudinal axis.

15. (canceled)

16. (canceled)

17. (canceled)

18. (canceled)

19. (canceled)

20. (canceled)

21. (canceled)

22. (canceled)

23. The surgical instrument of claim 1, wherein the drive member comprises a distal component for removably coupling to the surgical clip.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A surgical instrument for applying surgical clips to tissue, the instrument comprising:
an elongate shaft having a longitudinal axis;
an end effector on a distal end of the shaft including a pair of opposing jaws including a first jaw and a second jaw;
an internal channel extending at least from the elongate shaft to the end effector;
a wrist assembly coupling the end effector to the shaft, wherein the wrist assembly comprises:
a first articulation mechanism peripheral to the internal channel and configured to rotate the end effector about a first axis substantially perpendicular to the longitudinal axis;
a second articulation mechanism peripheral to the internal channel and configured to rotate the end effector about a second axis perpendicular to the first axis and the longitudinal axis; and
a drive member configured to translate longitudinal through the internal channel of the wrist assembly to deliver a surgical clip to the first and second jaws.

50. The surgical instrument of claim 49, wherein the wrist assembly defines a longitudinal centerline and the second articulation mechanism comprises first and second cross links extending from a first side of the longitudinal centerline to a second side of the longitudinal centerline.

51. The surgical instrument of claim 50, wherein the first cross link comprises a first pivot pin pivotally coupling the first cross link to the shaft and a second pivot pin pivotally coupling the first cross link to the end effector, wherein the second cross link comprises a third pivot pin pivotally coupling the second cross link to the shaft and a fourth pivot pin pivotally coupling the second cross link to the end effector.

52. The surgical instrument of claim 51, wherein the second articulation mechanism further comprises first and second cables extending through the shaft, the first and second cables peripheral to the internal channel, wherein the first and second cables are respectively coupled to the first and second cross links and configured to rotate the end effector relative to the shaft.

53. The surgical instrument of claim 49, wherein the first articulation mechanism comprises first and second force transmission lines extending from the shaft to the end effector, the first and second force transmission lines peripheral to the internal channel and configured to rotate the end effector about the first axis.

54. The surgical instrument of claim 53, wherein the first cable is configured to move the first and second opposing jaws into the closed position upon application of tension on the first cable, and the second cable is configured to move the first and second opposing jaws into the open position upon application of tension on the second cable.

55. The surgical instrument of claim 49, wherein the drive member comprises a distal component for removably coupling to the surgical clip.
